(19) United States
(12) Patent Application Publication
(10) Pub. No.: US 2022/0392598 A1
(43) Pub. Date: Dec. 8, 2022

(54) PRECISION SKINCARE SYSTEM AND METHOD

(71) Applicant: COMPASS BEAUTY, INC., Pleasant Hill, CA (US)

(72) Inventors: Chaz GILES, Pleasant Hill, CA (US); Troels MARSTRAND, Pleasant Hill, CA (US); Kana PANCHMATIA, Pleasant Hill, CA (US); Lieve DECLERCQ, Pleasant Hill, CA (US)

(73) Assignee: COMPASS BEAUTY, INC., Pleasant Hill, CA (US)

(21) Appl. No.: 17/230,653

(22) Filed: Apr. 14, 2021

Related U.S. Application Data
(60) Provisional application No. 63/125,685, filed on Dec. 15, 2020.

(51) Int. Cl. G16H 20/10; A61B 5/00; G06N 20/00

(52) U.S. Cl. CPC G16H 20/10; A61B 5/443; A61B 5/7264; A61B 5/442; A61B 5/0077; A61B 5/0075; G06N 20/00

(57) ABSTRACT

A system and method for precision skincare that uses a set of skin measurements to generate a skin profile and a skin need and the skin need is used to identify and predict an optimal skincare product formulation customized for each user based on the skin need of the user. The optimal skincare product formulation may include one or more active ingredients selected based on the skin profile and skin need of the user and a delivery mechanism for the one or more active ingredients. The optimal skincare product formulation may be generated using machine learning techniques and may include outcome feedback that optimizes the machine learning models.

### PRIORITY/RELATED APPLICATIONS

This application claims priority and the benefit under 35 USC 119(e) to U.S. Provisional Patent Application Ser. No. 63/125,685, filed Dec. 15, 2020 and entitled “Precision Skincare System and Method”, the entirety of which is incorporated herein by reference.

### FIELD

The disclosure relates to a system and method for skincare recommendations and skincare formulation and in particular to a skincare recommendation and skincare formulation system and method that measures a skin profile, generates a skincare formulation based on the skin profile, measures the outcomes of the generated skincare formulations and reassesses the generated skincare formulation based on the measured outcomes. The system and method may utilize artificial intelligence, machine learning and learning loops.

### BACKGROUND

Today skincare is a trial and error approach, where individuals must navigate over 5000 products by guessing at their skin needs, guessing what ingredients and products are best suited for their individual needs, and then guessing if the products are actually (measurably) working to improve their skin. Similarly, skincare manufacturers create skincare products based on small-based population mean studies and claims, which assume that each individual will respond to the active ingredients and delivery systems similar to the population mean. It has been shown empirically that this assumption is not accurate because the consumer and patient population is far more diverse than the small based population from which the mean was derived. Further, it is known empirically that individual responses to active ingredients and delivery systems vary significantly across individual consumers and are not accurately represented by population mean needs and claims.

The skin of each person is thus unique and each individual user's skin has, and can be assessed by, a set of skin parameters whose values indicate the health and appearance of the skin of the user. These skin parameters for a user can be combined into a skin profile. The skin profile of the user can be used to identify one or more of the underlying skin need(s) which must be affected in order to improve or maintain the overall health and appearance of the skin. The overall improvement in the appearance of skin could, for example, be measured by skin radiance, a clinical skin appearance measure that is used to assess the overall beauty and health.

Each skincare product has one or more active ingredients (“actives”) and a particular delivery mechanism. Each active ingredient, based on known literature, research, and empirical outcomes, may affect one or more of the skin parameters and thus address one or more skin need(s) and improve the radiance of the skin of the user. It is well known that a particular set of active ingredients in a skincare product and the particular delivery mechanism may improve the skin need(s) of the user. Given the individual needs of a user's skin, it is desirable to be able to custom formulate a skincare product for a user that improves the skin need(s) of that user and improves the radiance of the skin of the user. To avoid the trial and error approach that results from the current population mean approach to skincare product sales, it is desirable to identify the root cause of the skin need(s) and then be able to formulate a skincare product with one or more active ingredients that treat the need(s) of the particular user and a delivery mechanism that is most efficacious for the skin profile of the particular user.

In order to be able to determine the root cause of the skin need(s) for a particular user, it is necessary to measure a plurality of different skin parameters and sub-parameters that provide a way to evaluate a current skin radiance of the user and identify the skin need(s) of the user. Hardware devices exist that can measure different parameters of the skin, but no system exists that measures the particular combination of parameters that indicate skin radiance based on the set of parameters in the skin profile. It would be desirable to be able to accurately and efficiently measure the skin radiance of a user which may be used to assess the skin need(s) of the user.

Some known systems may permit one or more skin parameters to be measured in person, but no systems today measure the plurality of measures required to accurately assess skin appearance and health at the surface and sub-dermally. Further, these in person systems do not provide a way to leverage those personal measurements for remote skin measurements thus limiting their usefulness for consumers and patients. Current remote skin measurements, such as using a mobile device, cannot measure the set of parameters that can be measured in the laboratory. Thus, it is desirable to be able to infer the skin profile of the user with the one or more skin parameters from the remote skin measurements since the remote skin measurements allow a user to gather those remote skin measurements, such as for example at home, with their mobile device, those remote skin measurements can be mapped to the skin profile and a custom formulation for a skincare product can be determined even with only the remote skin measurements. Systems today cannot achieve this result and thus consumers are not able to avoid the current trial and error process.

It is desirable to be able to custom formulate a skincare product (with one or more active ingredients and a delivery mechanism) that specifically addresses the skin need(s) of the user. Today, consumers may seek recommendations from trained individuals like a dermatologist. While a dermatologist is trained to identify skin need(s) and can recommend ingredients for skin care, they are constrained by the lack of multivariate assessment devices, and the sheer volume of ingredients, products, response curves and thus cannot scaleably or reliably assess, recommend, or formulate the optimal individual formulation for each particular user. Thus, it is desirable to be able to formulate a custom skincare product with one or more active ingredients and a delivery mechanism customized to the user that is not available today.

Today, a user may purchase or receive a new skincare product and try it. The user may visually notice whether the skincare product is achieving its goal of addressing the skin need. The user may also go to a dermatologist to remeasure a subset of parameters and the dermatologist can recommend a different skincare product. However, there is no system today that can remeasure of the set of skin parameters of the user after the skincare product has been used and then automatically provide an updated skincare product formulation to the user in response to the remeasured set of skin parameters.

It is desirable to be able to re-measure the one or more skin parameters of the user after use of the skincare product and generate data about the outcome of the skincare product use by the particular user. For example, for a certain skin issue, it may be known that a certain value of a skin parameter causes and/or indicates the skin issue so that the remeasurement of that certain skin parameter after use of the skincare product empirically shows whether or how well the customized skincare formulation is working. This would allow the customized formulation to be adjusted as needed based on the outcome. The adjustment may be because the current formulation is not working effectively, is not working effectively for that skin profile, or because the skin parameters of the user have changed over time. No known system accomplishes this remeasurement of the skin parameters and adjustment of the customized skincare formulation of the user.

It is desirable that any skincare formulation is as customized as possible to provide the optimal results for each user and each user skin profile. However, existing systems fail to assess the parameters to determine the root cause, assess the efficacy of each particular formulation for each skin profile and thus do not learn and do not become better at determining the optimal customized skincare formulations. It is desirable to provide a system that can make customized skincare formulation recommendations and use the outcomes of each user to improve the customized skincare formulation recommendation process for that individual user and all other users. This does not happen today.

### BRIEF DESCRIPTION OF THE DRAWINGS

FIG. 1A illustrates an example of a cloud based precision skincare system;

FIG. 1B illustrates a method for precision skincare;

FIGS. 2A and 2B1-2B3 illustrate a physical precision skincare process;

FIGS. 3A and 3B1-3B4 illustrate a remote measurement precision skincare process;

FIGS. 4-1 and 4-2 illustrate more details of the machine learning predictive engine process;

FIGS. 5A1-5A2 and 5B1-5B2 illustrate the overall precision skincare process divided into user facing processes and backend processes; and

FIGS. 6 and 7 illustrates example of the user interface of the skincare system showing a recommended formulation and skin profile for each user.

### DETAILED DESCRIPTION OF ONE OR MORE EMBODIMENTS

The disclosure is particularly applicable to a system and method that provides skincare recommendations and it is in this context that the disclosure will be described. It will be appreciated that the system and method has greater utility, such as to recommendations for other healthcare products or services and the system may be implemented in other ways or with other architectures that are all within the scope of this disclosure. As examples, the system and method disclosed below can also be used to assess and recommend custom hair products based on a hair profile and hair measurements, makeup products or nutrition products. Furthermore, the system and method disclosed below can be used for dermatology pharmaceutical development/treatment or injection/oral ingestible treatments of skin. It is understood that for each different use case, a different set of measurements may be captured and then used to make the recommendation for the particular use case and those modifications are understood and within the scope of the disclosure.

### Description of the Details of a Specific Implementation of the System

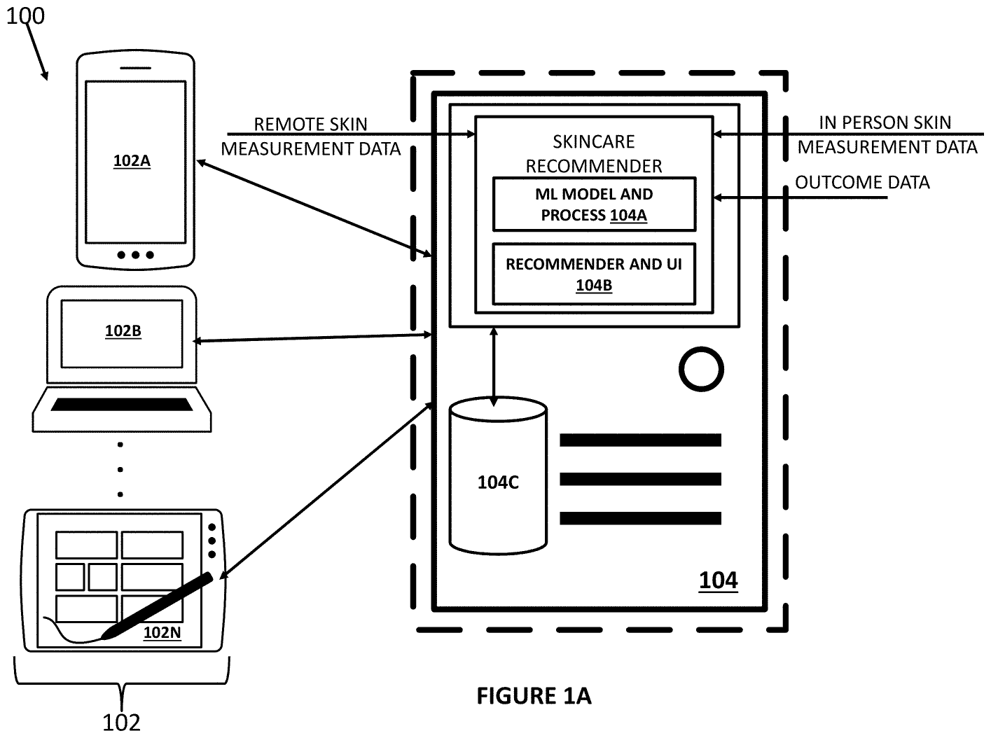

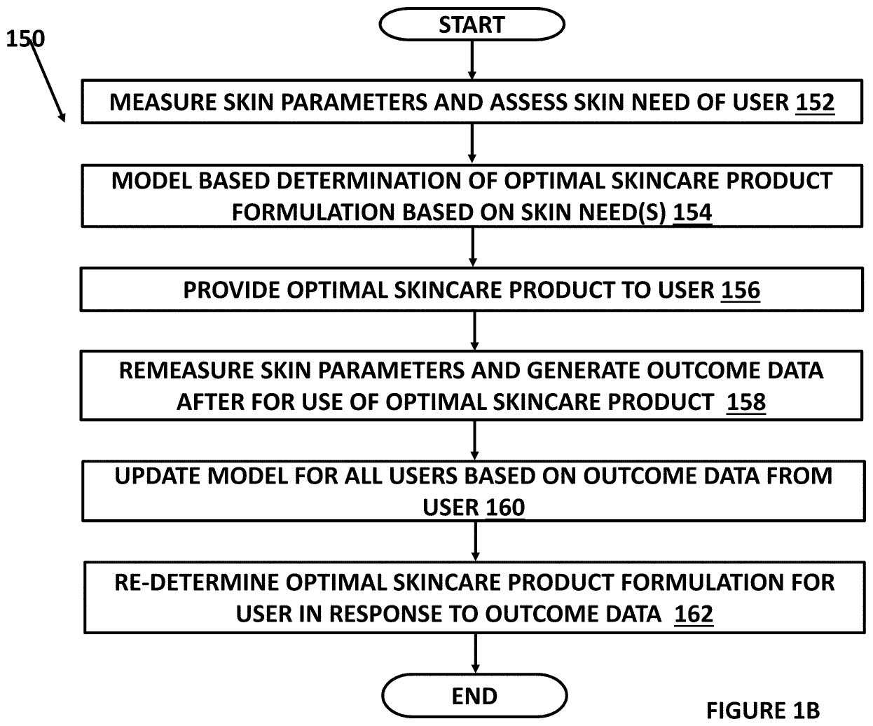

FIG. 1A illustrates an example of a cloud based precision skincare system 100 that may be used to provide precision skincare to each user based in part on the measurements of a set of skin parameters of the user. The set of skin parameters of the user may be a skin profile and may be used to determine a skin need of the user. The skin need of the user is an aspect of the skin of the user that is deficient and/or indicates the underlying root cause of a particular skin concern or issue for the user. The precision skincare system may be used to create one or more skincare products in which the set of skin parameters and skin need are used in a machine learning process to select one or more active ingredients and a delivery mechanism for each skincare product that is an optimal skincare product for the user that improves the skin need of the user. The system may also produce that optimal skincare product and provide it to the user. The system may also remeasure the set of skin parameters of the user after use of the optimal skincare product to generate outcome data that is used to improve the machine learning process.

The system 100 may be implemented as a cloud based architecture in which most of the backend 104 components are implemented using cloud computing resources and the engines and processes of the backend 104 may be each implemented as a plurality of lines of instructions that are executed by a processor of a cloud computing resource so that the processor is configured to perform the processes of the precision skincare system as discussed below. The backend 104 may be located in a third party cloud site or data center or may be co-located with a physical location of the skincare company that uses the precision skincare to recommend skincare for its customers.

The system 100 may have one or more computing devices 102, such as devices 102A, 102B, . . . , 102N, used by users to interact with the backend system 104 using an app or a web interface wherein the interactions may include submitting remote measurement data, receiving data for displaying a skincare dashboard or the skin need(s) of the user or for ordering the optimal skincare product for the user. Each computing device 102 may have a processor, memory, display, user input device and wired or wireless communication circuits that permit the user to interact with the backend 100. For example, each computing device 102 may be the device for capturing the remote skin measurements of the user and the device used by the user to interact with the backend 104 and other websites. Alternatively, the computing device used for capturing remote skin measurements of the user may be different from the computing device used to interact with the backend 104 and other websites. For example, each computing device may be a smartphone device 102A, such as an Apple iPhone or Android operating system based device, a laptop computer 102B, a tablet 102N, a personal computer, a terminal and the like. Each computing device 102 may connect to interact with the backend 104 over a communications path as is well known.

The backend 104 may include a machine learning system 104A that includes a machine learning model and process that performs machine learning as part of the precision skincare process as described below. The backend 104 may also have a recommender and UI engine 104B that determines a skin need of the user, uses the machine learning model to select one or more active ingredients and a delivery mechanism for an optimal skincare product for the user that addresses the skin need of the user and generates the user interface provided to each user. The backend 100 may also have a store 104C that stores various data of the system including user data, the machine learning instructions, the ML model, the training data and the like in a hardware or software based database. The machine learning system 104A may also have one or more learning loops that optimize the model in order to provide better formulated optimal skincare products. The backend 104 may also include a facility that can produce the optimal skincare products and deliver those optimal skincare products to each user.

The backend 104 may operate in an in person mode (at a physical location) in which the set of skin measurements of the user are captured at the physical location (the in person skin measurement data as shown in FIG. 1A) and/or in a remote mode in which the skin measurements of the user are captured outside of the physical location (the remote skin measurement data as shown in FIG. 1A) and the skin measurement data from both modes of operation are fed into the system and used to perform the precision skincare process. At the physical location for the in person mode of operation, the system may have one or more pieces of hardware for capturing the set of skin measurements. For example, the pieces of hardware may include a skin impedance measurement device, a line-field confocal optical coherence tomography (LC-OCT) system, a mass spectrometry system, cross polarized and UV imaging devices, Spatial Frequency Domain Imaging hardware, Hyperspectral imaging hardware, RGB imaging hardware, Confocal Raman Spectrometry and Courage and Khazaka probes for measurements of, for example, Transepidermal water loss, Skin temperature, Skin pH, Skin hydration, Skin tone and/or Skin glossiness. Note that while the pieces of hardware may be commercially purchased, the combination of these pieces of hardware (and the software) have not been used to generate the set of skin parameters disclosed herein. For the remote mode, a user may use their computing device 102 (or a dedicated hardware device different from the computing device 102) to capture, for example, a red-green-blue (RGB) image of their skin (the remote skin measurement data in FIG. 1A) that is sent to the backend 104. In an alternative embodiment, the computing device 102 may include a hyperspectral chip or chips integrated into the computing device that can reconstruct or directly measure a hyperspectral cube from the skin of the user.

In some implementations of the system, the physical location may house all of the hardware to capture and analyze the set of skin measurements. In other implementations, the system may be a hybrid system that has hardware at the physical location to capture the set of skin measurements, handles interfacing to user-facing data presentation, and data exchanges and the backend 104 in the cloud and the backend 104 may consist of a series of data analysis services, robust data storage of both raw and processed device data. In the implementation, the software stack at the physical location may consist of standard browser based technologies for data exchange and data presentation, while the backend systems 104 may consist mainly of analytical tools written in python with one or more third party core libraries such as tensorflow, numpy, pandas, scipy and similar.

As shown in FIG. 1A, the backend 104 may include the cloud based storage system 104C that stores all collected skin measurements in both raw format as well as processed format. The processing of the skin measurement data may include standardization and normalization. The skin measurement data stored in the storage system 104C allows for easy retrieval of skin-profiles for a particular user or for a particular set of users with common skin profiles. The storage 104C may also store outcome data (described in more detail below with reference to FIG. 1B) that is a remeasured set of skin parameters for a user after the user has used the optimal skincare product. The outcome data stored in the storage 104C may be identical to skin measurement data stored except that the outcome data includes a given ‘treatment’ code specifying the user specific combination of one or more active ingredients and delivery mechanism in the optimal skincare product used by the user and the treatment code will be associated with the new set of skin measurements.

FIG. 1B illustrates a precision skincare method 150 for a user that may be performed using the system/platform 100 in FIG. 1A, but may be performed by differently configured systems that are still within the scope of the disclosure. The method first measures a set of skin parameters of the user (a skin profile) and assesses the one or more skin need(s) of the user (152) based on the set of skin parameters.

In this skin parameter measurement process 152, the method captures and measures the set of skin parameters using one or more measurement devices/systems (discussed below in more detail). The set of skin parameters of the user in this process 152 may be captured in several different ways.

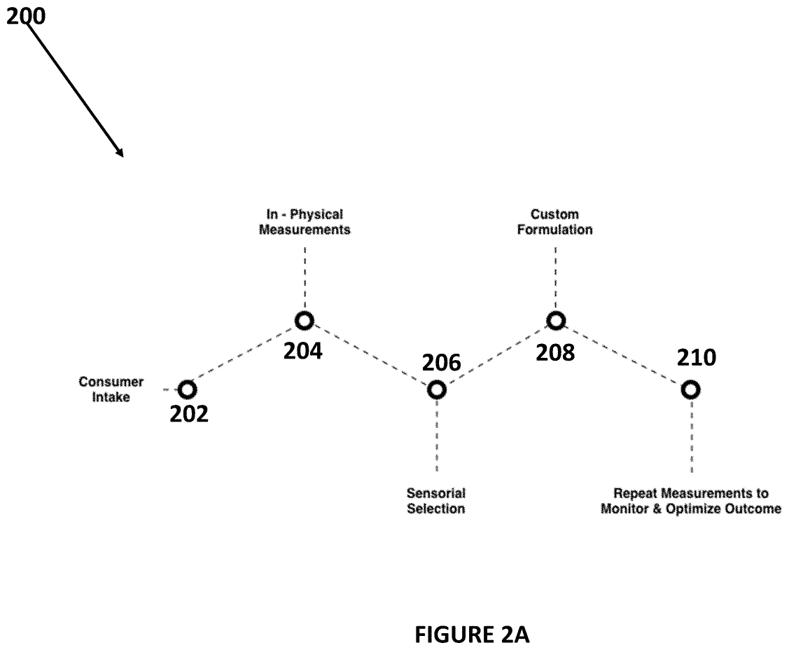

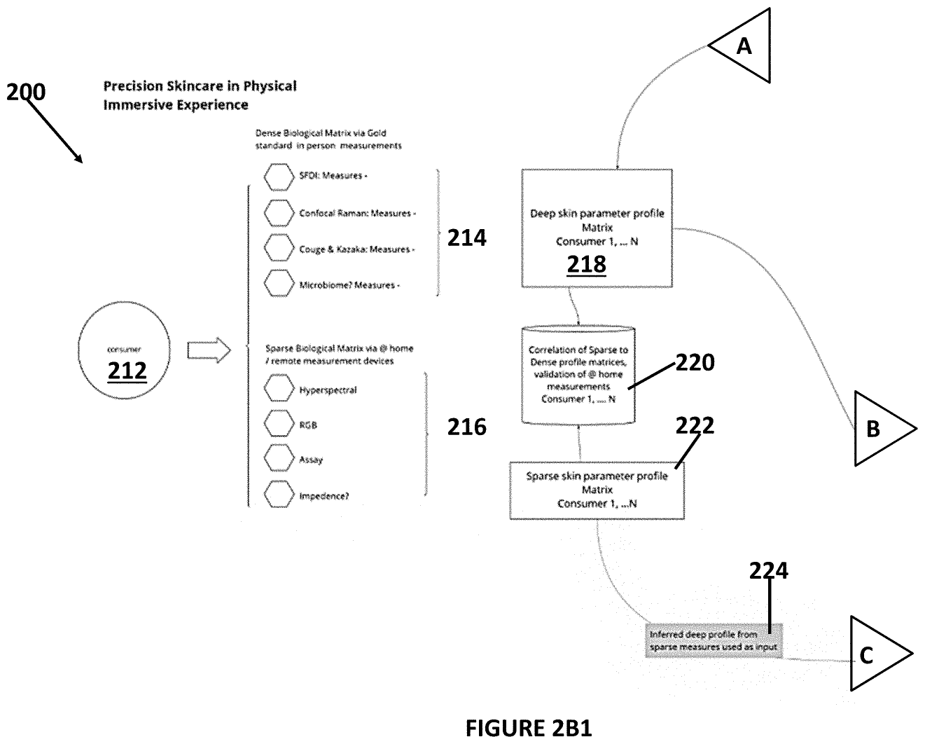

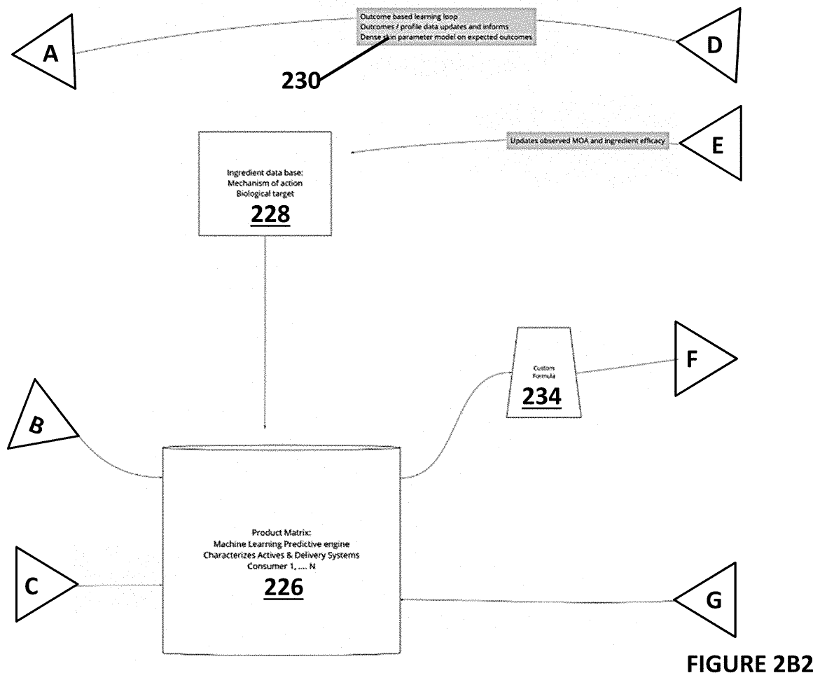

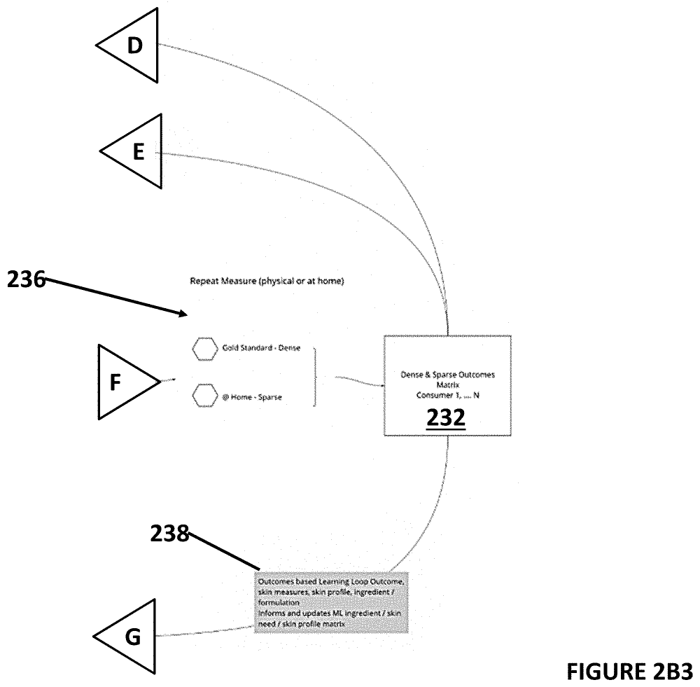

First, the set of skin parameters may be captured during an in person measurement process (FIG. 2A) so that a dense skin parameter matrix, as discussed below, is generated based on the in person measurements. The dense skin parameter matrix is then used to determine and provide the optimal skincare product(s) for the user.

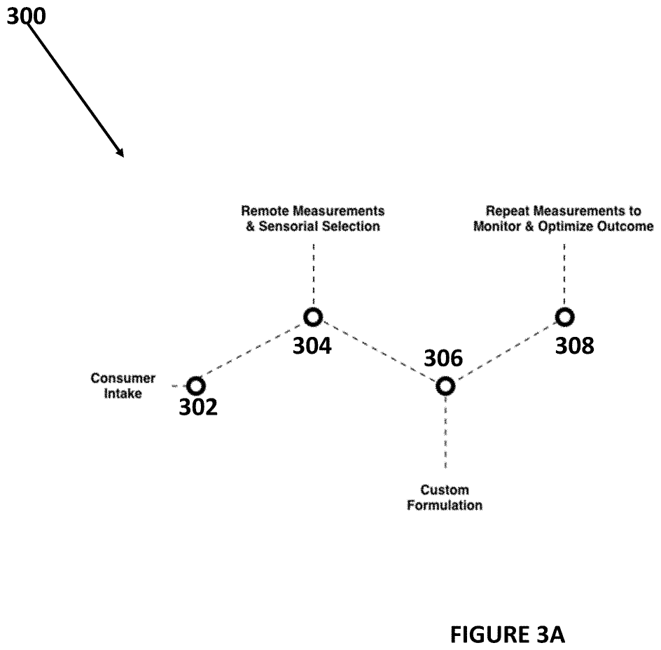

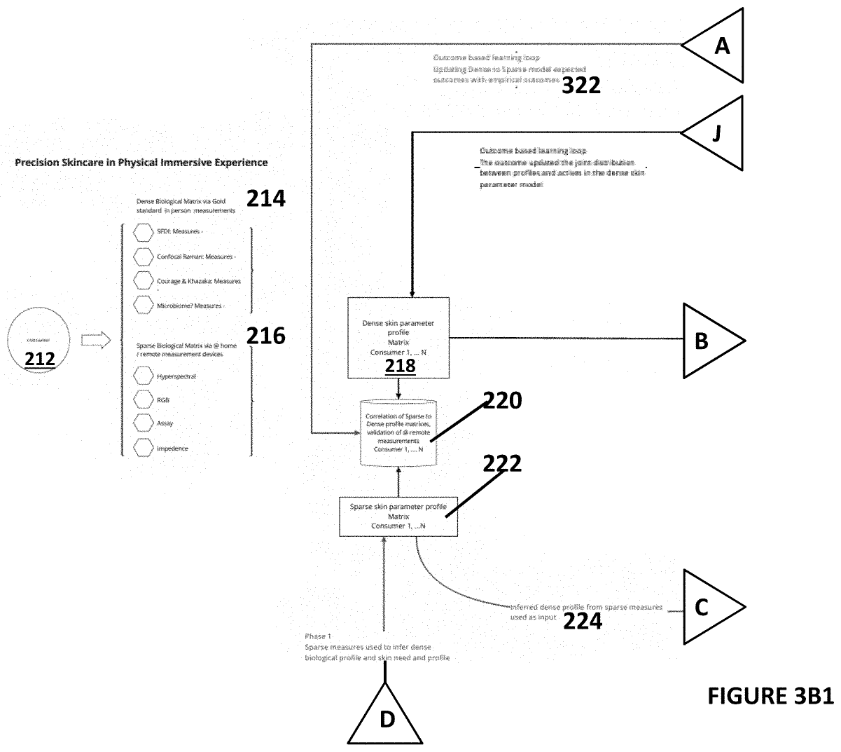

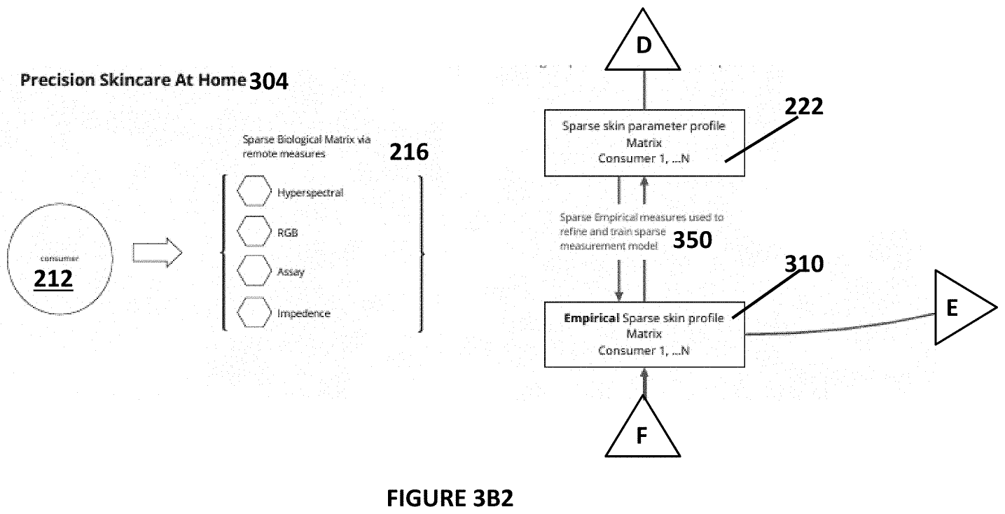

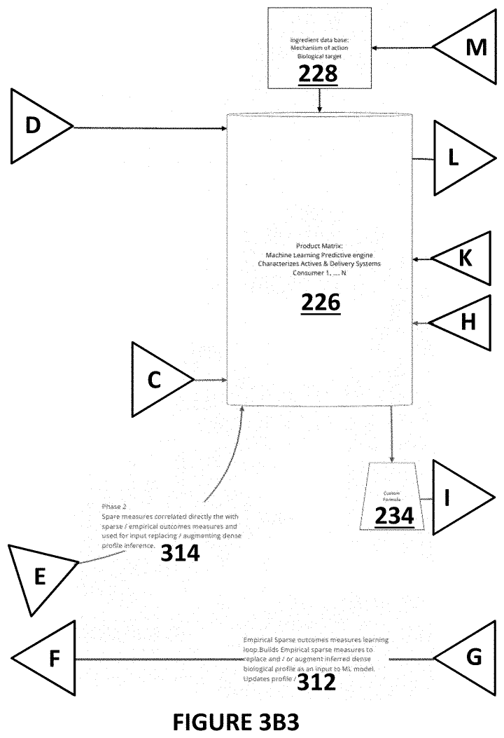

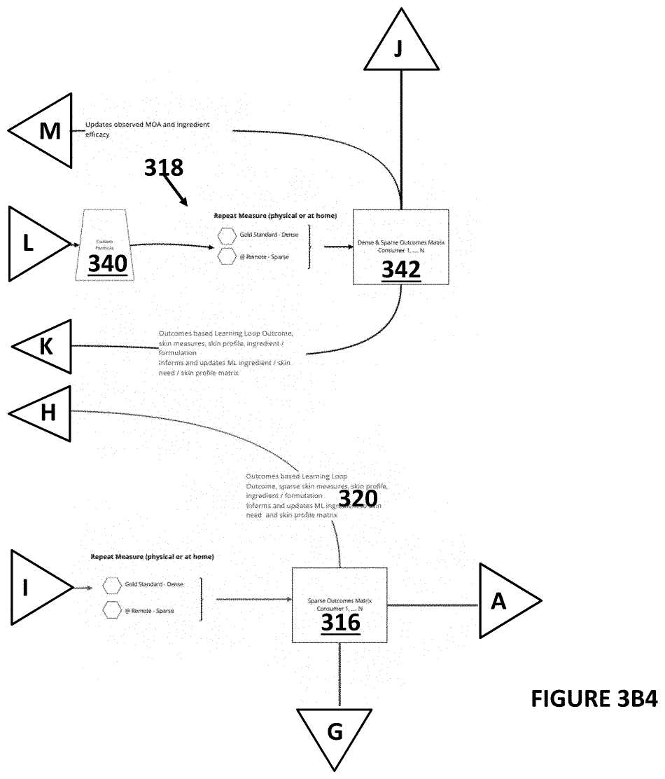

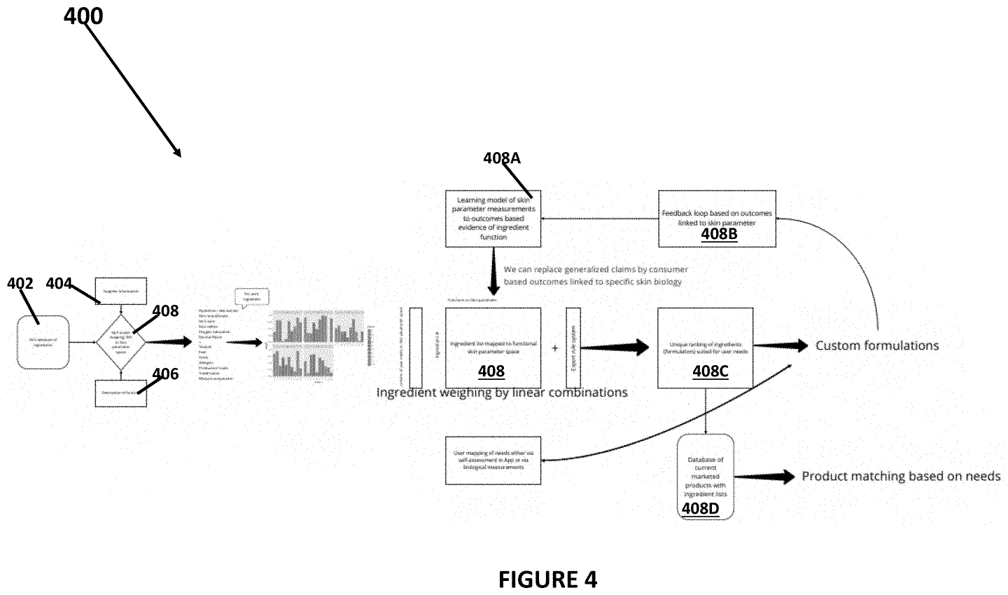

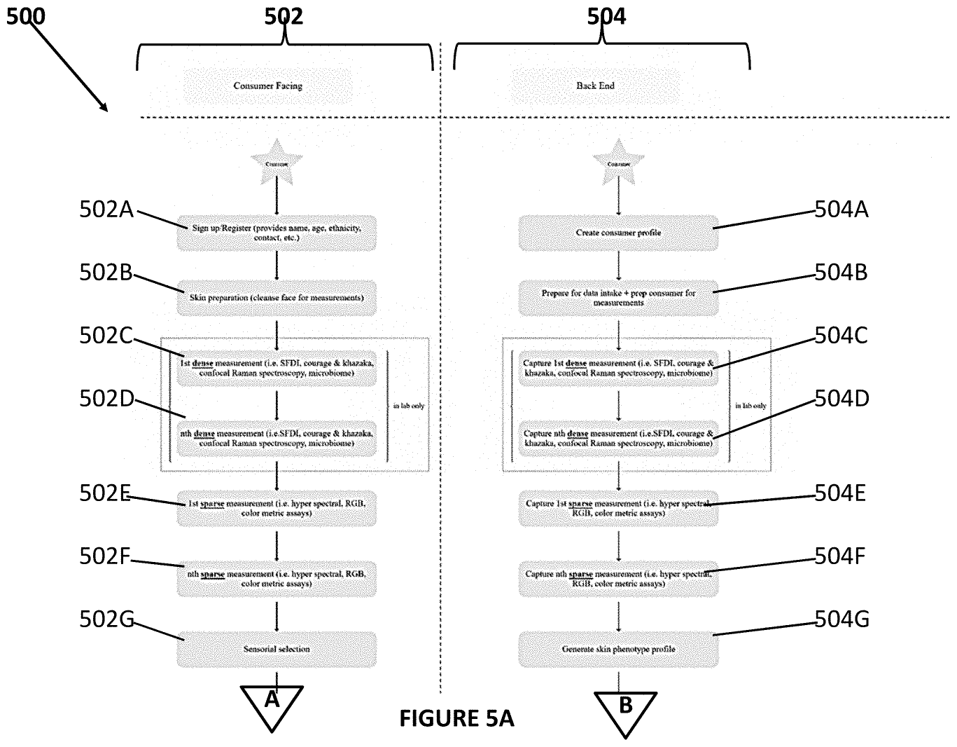

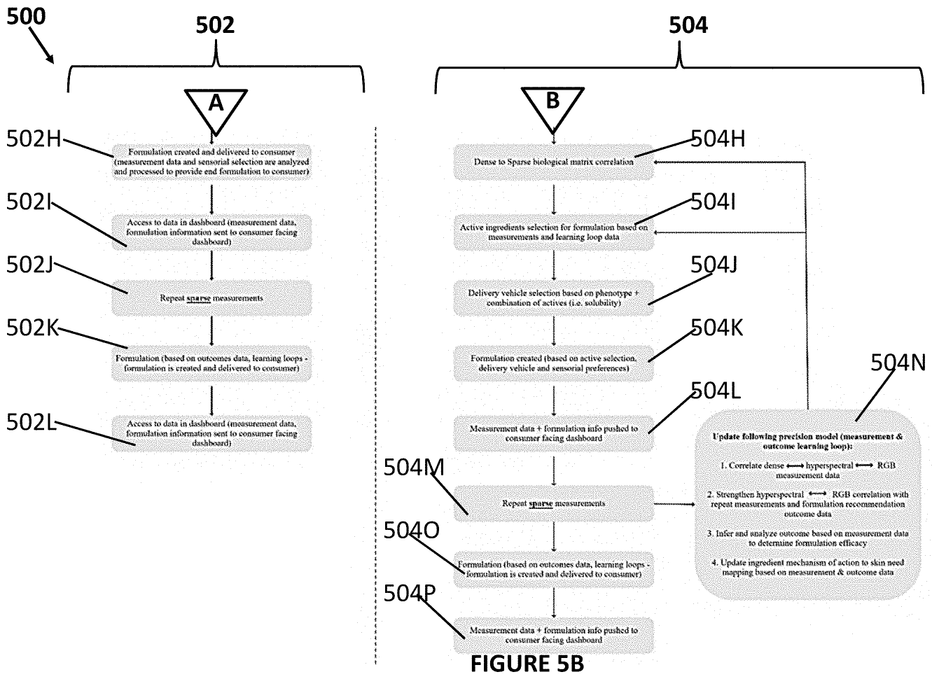

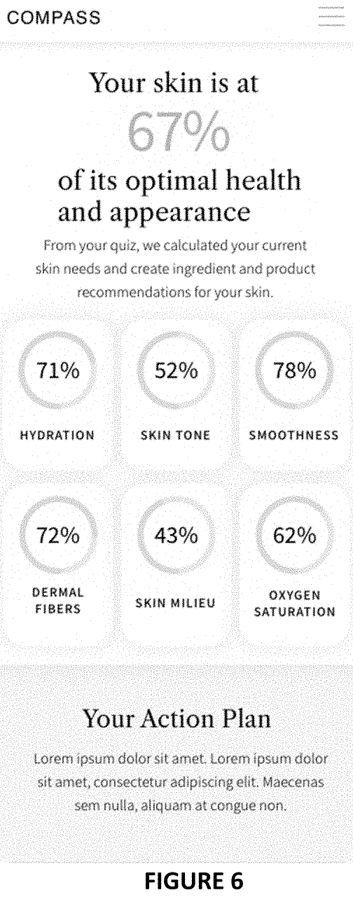

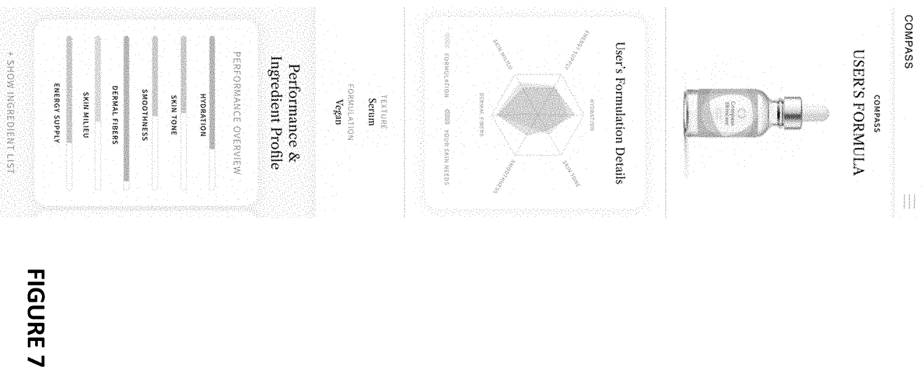

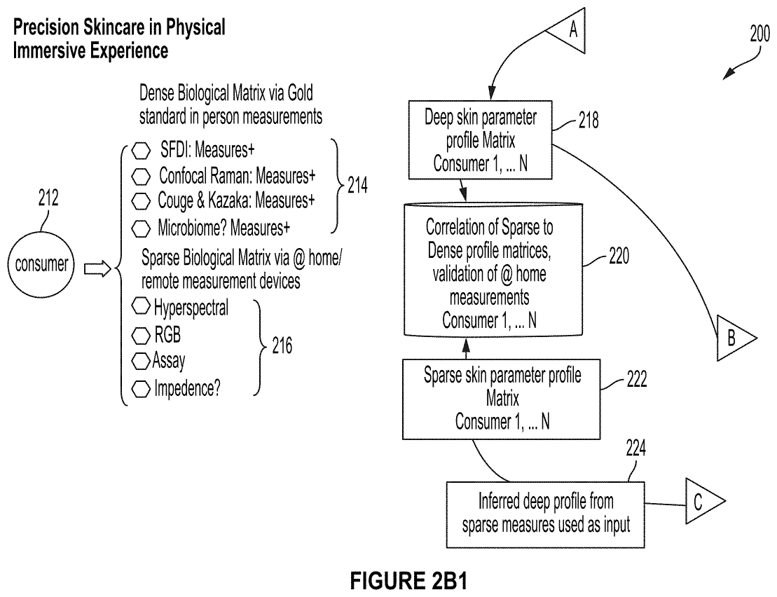

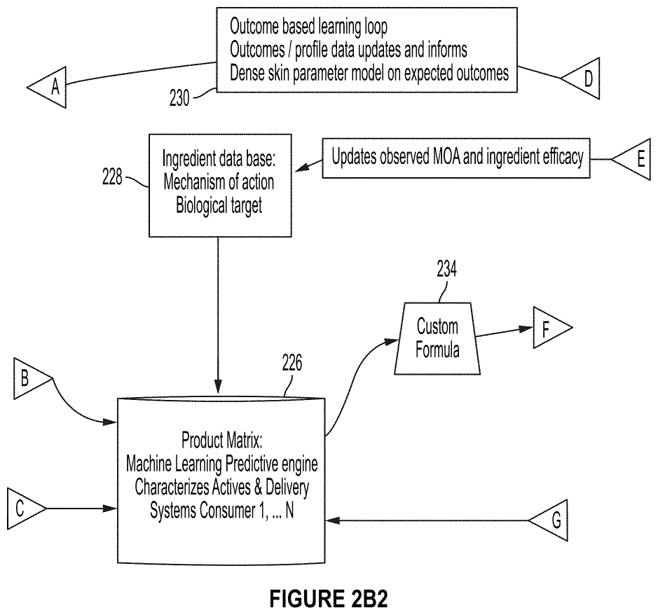

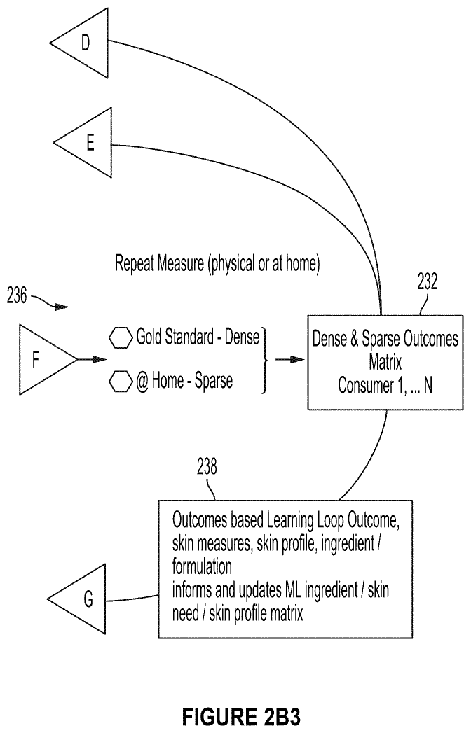

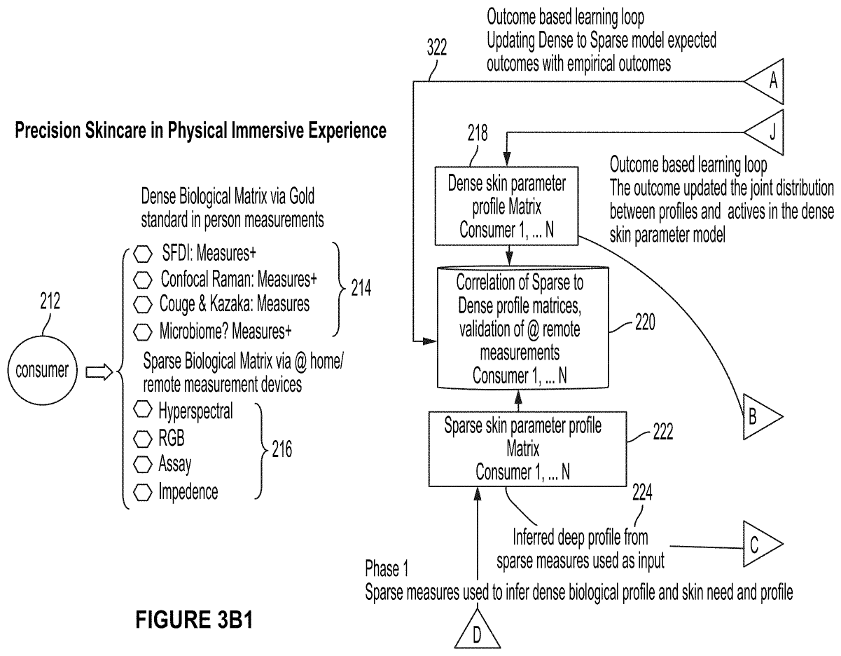

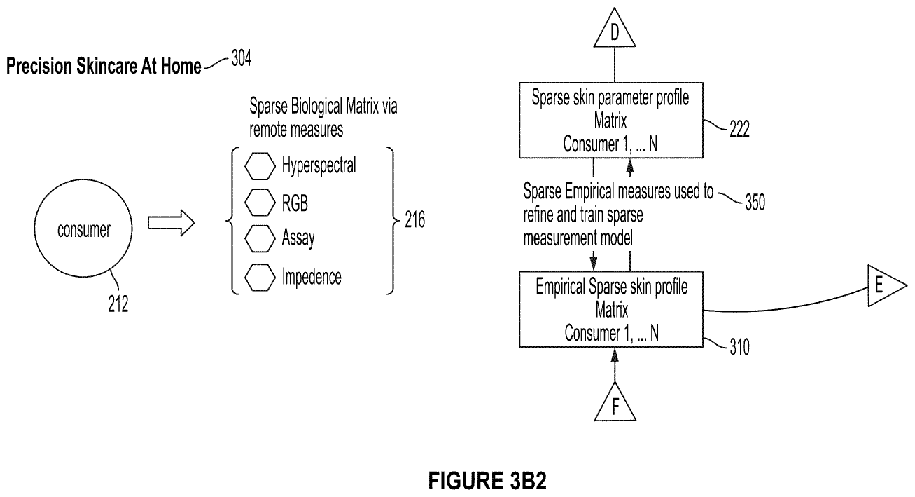

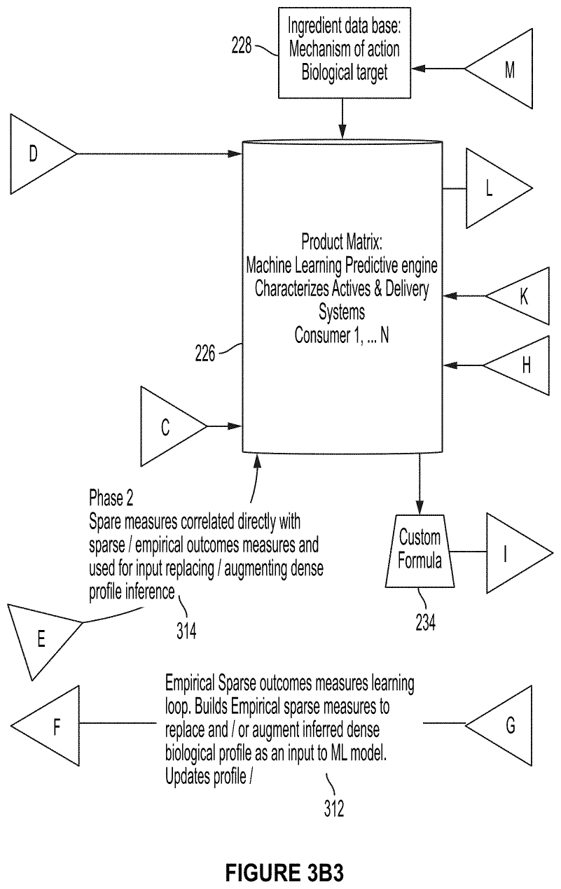

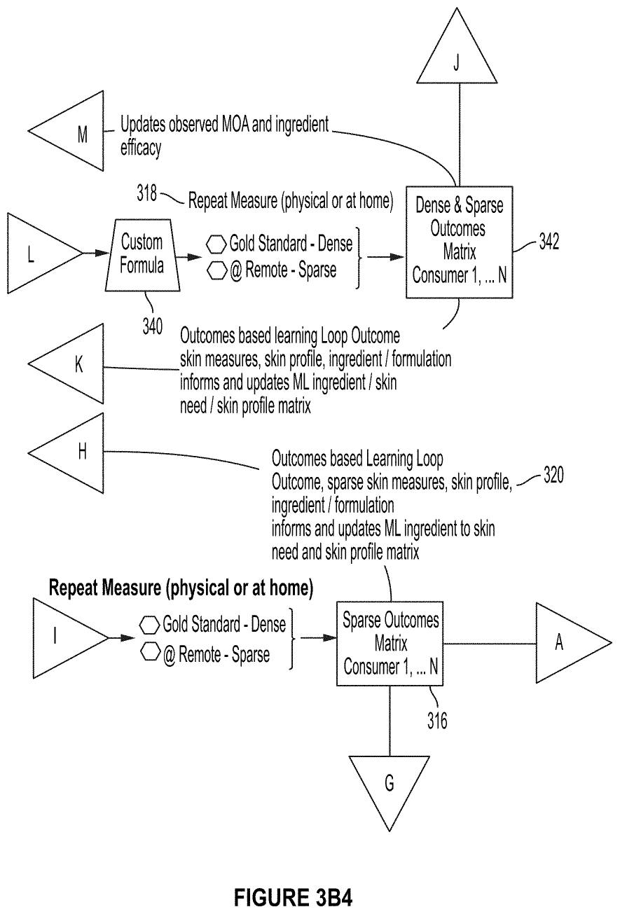

Second, the set of skin parameters may be captured during both the in person measurement and during a remote measurement (FIG. 3A). In this case, the dense skin parameter matrix and a sparse parameter matrix (both of which are discussed below) may be generated. The dense skin parameter matrix is then used to determine and provide the optimal skincare product(s) for the user.

Third, the set of skin parameters may be captured during only the remote measurement process to generate the sparse skin parameter matrix. The sparse skin parameter matrix is used to infer the dense skin parameter matrix of the user. The dense skin parameter matrix is then used to determine and provide the optimal skincare product(s) for the user. It is important to note that the precision skincare system and method can determine and provide optimal skincare products using all of the above ways that skin parameters are captured.

The skin parameters may be used to generate a skin radiance assessment. In a preferred embodiment, the set of skin parameters may be six skin health measures which, together, drive the overall radiance measure of skin. The health measures are a multivariate set of measures that roll up to the singular objective function of skin radiance. The set of skin parameters may include: Skin barrier/Hydration (including Barrier defects, Water retention/transportation and/or Cell proliferation); Smoothness (including Lipid combination and organization, Desquamation and/or Pore size and clogging); Skin tone (including Melanin and pigmentation, Chromophores and/or Genetic/epigenetics); Skin Mileu (including Microbiome, Micro vasculature and/or Local immune system); Energy Supply (including Hemoglobin/deoxyhemoglobin, Mitochondrial health and/or Pollution); and Dermal fibers (including Collagen & elastin matrix, Fibroblast proliferation and/or Wound healing capacity). No current system measures each of the above skin parameters to generate the skin radiance that provides a skin profile of the user. In turn, the skin profile of the user may be used to determine one or more skin need(s) of the user.

Another novel aspect of this skin parameter measurement process 152 is the remote measurement experience of the user as shown in FIG. 3A and described below. In the remote measurement experience, the user may use a measurement device that captures a limited set of data about the skin of the user. The method then maps/correlates these limited set of data about the skin from the remote measurement of the user to a model that contains the aggregate, full set of skin parameters that are measured during an in person measurement process. Thus, even with the limited set of data about the skin, the method is able to still assess the skin need(s) of the user. This novel remote measurement process means that the subsequent processes of the precision skincare method (including recommending the optimal skincare product to the user) can be performed even when only the limited set of skin data from the remote measurement are used and thus the method does not require every user to do the in person measurement process.

In more detail, the set of skin measurements from an in person measurement is a full biological measure matrix of measurements for a given user that may be known as a dense matrix. The limited set of skin data from a remote measurement, such as an RGB image, may be known as a sparse matrix. To map/correlate the limited set of data from the remote measurement to the dense matrix, the method may use an inference method. For example, in one embodiment, the method may infer the set of parameters in the dense matrix by reconstructing a hyperspectral cube from the RGB image. Alternatively, if the computing device 102 includes hyperspectral chips, then the hyperspectral cube may be reconstructed from the output signals from the hyperspectral chips or directed measured from the skin of the user. For example, this process 152 uses full biological measures paired with hyperspectral images and RGB images of the same user measured at the physical location and then perform the correlation by mapping prior observed hyperspectral images of the user to full biological measures and/or by an interpolation between multiple prior observed hyperspectral images of the user to the full biological measures.

In more detail, the mapping/correlation of the limited set of skin data from the remote measurement to the full set of skin parameters from the in person measurement, a two-phase approach may be used. First, a reconstruction algorithm of Voyager81 may be used to infer the hyperspectral image from the RGB image or the hyperspectral cube may be reconstructed from the hyperspectral chip data in the computing device or measured directedly from the skin using the hyperspectral chips. With this reconstructed hyperspectral image, the system can use the linked hyperspectral/dense measurement pairs from users during the in person measurement to infer the most likely dense measurement from the remote measurement using either a nearest neighbor approach or a more complex modelling approach where the dense matrix is inferred as a linear combination of the spectral features of the dense matrix. Since this approach involves two inference steps (RGB to hyperspectral, hyperspectral to dense) and each step may have a loss of accuracy, the system may have a secondary approach directly linking the inferred hyperspectral data to product recommendation and outcomes. Since the remote measurement process will do re-measurements using the same hyperspectral inference process, the process is able to collect empirical data linking initial RGB images (and inferred hyperspectral images) directly to a given product recommendation and its user specific outcomes. And then similarly, as in the in person measurements, the remote measurements enable the model to create the joint distribution between measurements and product recommendations directly to outcomes measured on the same scale as seen in 310 in FIG. 3B2. In other words, where the in person measurement experience starts with an expert system prior to being fueled with empirical outcomes, the remote measurement model starts with an extra inference step to previous skin profiles, prior to having enough data to enable its own end-to-end learning model.

Using the skin need(s) of the user (generated through the in person skin measurements or the remote skin measurements as described above), the method may determine an optimal skincare product formulation for the user (154). The method may use a model based machine learning to programmatically determine the optimal skincare product formulation customized for the user wherein the optimal skincare product formulation customized for the user has one or more selected active ingredients and/or a selected delivery mechanism. The one or more active ingredients (and quantities of each active ingredient) and the delivery mechanism are selected because together they are predicted, or observed, to be the most efficacious formulation to treat or improve the skin need of the user. The model may have a plurality of entries and each entry has a particular skincare active ingredient, the one or more skin need(s) that are affected by that active ingredient and a confidence score. The model may also have an entry for each delivery mechanism, a skin profile (based on the set of skin parameters) for which the delivery mechanism can deliver the active ingredients and a confidence score. Using these entries in the model and based on the assessed skin need(s) of the user, the method automatically selects the optimal skincare product formulation for the user that includes one or more active ingredients (including a level or amount of each active ingredient) and the delivery mechanism.

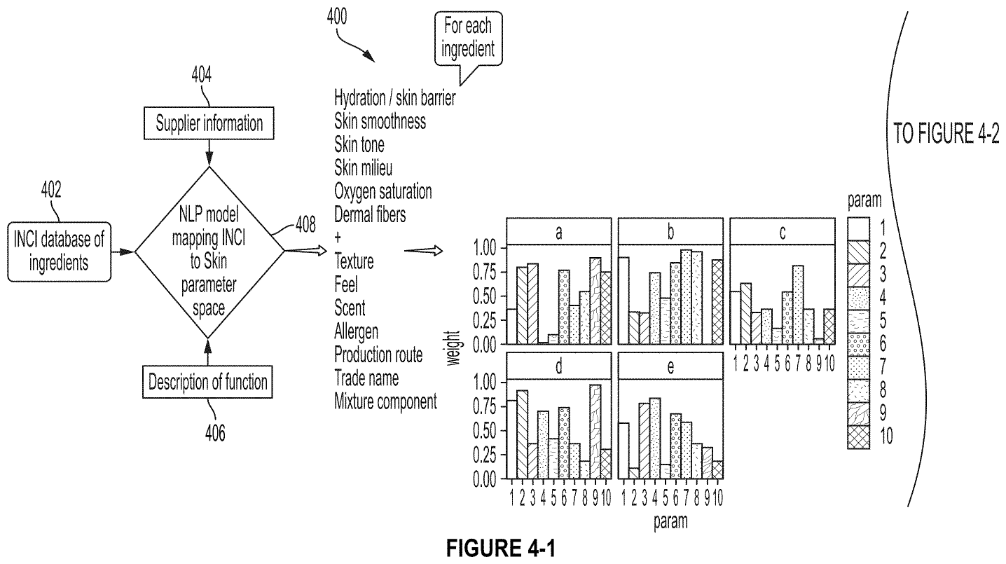

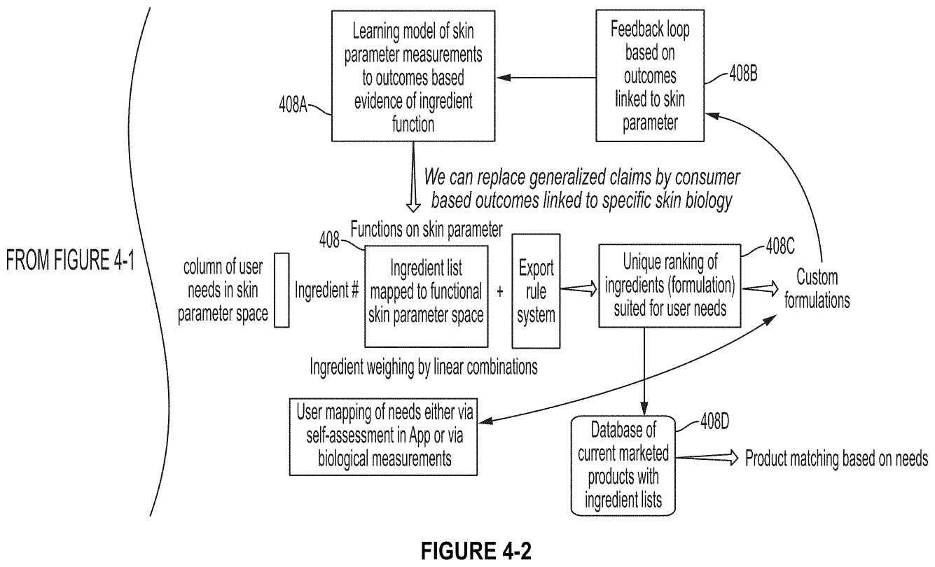

To generate the custom formulations, the method performs a selection of the active ingredients for the custom formulation and amounts of each active ingredient based on the set of skin parameters, skin profile, and/or user preferences. For example, the dense skin measurements (based on the in person skin measurements) may be used as inputs into a product ingredient matrix machine learning (ML) model (described below with reference to FIGS. 4-1 and 4-2 in more detail). The ML model analyzes the individual skin need scores (based on the skin measurements), assigns relative weights to the individual skin needs for the particular user and creates a priority ranking of the skin needs for the particular user. Based on the skins needs and priorities, the ML model evaluates the possible active combinations, considering their delivery mechanisms of action, biological targets, phenotypic efficacy, and their predicted outcomes for that user's skin need and profile. The ML process then creates the optimal skincare product formulation customized for the user in which the one or more active ingredients (and amounts of each active ingredient) are selected from a set of active ingredients known to be effective for the user's specific skin needs. To generate the optimal skincare product custom formulations, the method also performs delivery vehicle selection in which the ML model selects the optimal delivery system among a set of delivery systems.

The optimal skincare product formulations selected by the method may be for various skincare products. For example, the optimal skincare products may include a cleanser, a treatment product, a toner, or a moisturizer. However, the method may be used to select optimal skincare product formulations for various other skincare products. Furthermore, the method could be used to produce optimal product formulations for any other products such as shampoos or hair care products or other products in which an optimal product formulation may be desirable.

The method may then produce the optimal skincare product formulation for the user (156) with the selected one or more active ingredients, the corresponding level or ratio of each individual active ingredient and the selected delivery mechanism. To produce the optimal skincare product, the optimal skincare product formulation may be sent to either 1) a formulation chemist or 2) an automated production machine or 3) a combination of both, that combines the appropriate active ingredients at the designated levels, includes or adds the active ingredients to the designated delivery mechanism and then mixes, heats, and dispenses or fills formulation into the final packaging. The method may then deliver the optimal skincare product to the user.

After the user has used the optimal skincare product for a period of time, such as 3 months, a year or any other period of time, the method remeasures the set of skin parameters of the user and generates outcome data based on the use of the optimal skincare product by the user (158). For example, the outcome data may indicate that a particular active ingredient to a particular user with a particular skin need and particular skin profile did not improve the skin parameter associated with the skin need and the entry for that particular active ingredient may be adjusted in the model to reflect that outcome data. As another example, the set of skin measurements of the user will change over time as skin is a dynamic biological organ reacting to stress, hormones, aging of the user, a new physical environment of the user (different location with different altitude, pollution, different exposure to sun), etc. and the model with the active ingredients and delivery mechanism should be updated to reflect the changes to the set of skin measurements of the user. As yet another example, the use of the optimal skincare product may address the skin need of the user which can be measured by the new set of skin measurements and an amount of an active ingredient in the optimal skincare product may be decreased for a new optimal skincare product based on the new set of skin measurements. As another example, the precision skincare system may recommend a skincare product with particular active ingredients to improve a first skin parameter (that may be the most concerning skin parameter) of the user, and, when that first skin parameter has been improved (as shown by the remeasured skin measurements), recommend a skincare product with possibly different active ingredients to improve a second skin parameter and so on. In one embodiment, the set of skin measurements may be remeasured using the remote measurement process described above and then the new set of skin measurements may be inferred as described above. Alternatively, the in person process may be used to measure the new set of skin parameters.

The method may then update the model (160) based on the outcome data which may be known as a learning loop. In the example above, the entry for that particular active ingredient may be adjusted in the model to reflect that outcome data. For example, the entry may be adjusted to indicate less confidence that the particular active ingredient addresses the particular skin need for that particular skin profile as shown by the skin parameters associated with the skin need. Note that the updating of the model based on the user's particular outcome data benefits the user (since the system can re-predict the custom formulation of the optimal skincare product for the user based on the outcome data) and all other users since the model is updated and thus can be used for all of the other users of the method. Thus, this updating of the model based on the outcome data is a learning loop that optimizes the selection of the one or more active ingredients and the delivery mechanism using the model for the optimal skincare product customized for each user.

In more detail, the method may update the joint distribution between skin parameter measurements and user optimal skincare product formulation with the observed outcome data. This joint distribution is continuously updated with each new piece of outcome data evaluating the posterior probability distribution of a beneficial outcome for skin parameter X given a baseline skin profile Y and a product formulation Z. Either can be done in the multivariate space for singular skin parameters and/or a combination thereof, essentially leading to a linear combination of the skin parameter inputs. The outcome data and its empirical data may or may not entirely overwrite the model with respect to a new optimal skincare product formulation for a particular user. For each new selection of the optimal skincare product formulation, the entirety of all observed outcomes at the time of the new selection of the formulation are evaluated with respect to a new skin profile of the user, as well as any outcome data for the particular user.

For the particular user for which the outcome data is generated in the remeasuring process 158, the method may then re-select the one or more ingredients (and amount of each active ingredient) and the delivery mechanism for the optimal skincare product formulation for the user using the process 154 described above and in response to the outcome data (162) and deliver that updated optimal skincare product to the user. Note that the processes of remeasuring the set of skin parameters, updating the model and re-determining the optimal skincare product formulation for the user may occur repeatedly for each user. Thus, the model becomes more optimized for all users with each piece of outcome data and also continuously adapts and changes the optional skincare product formulation for each user as the skin parameters of the user change over time for the various reasons described above.

FIG. 2A illustrates more details of the user experience during the in person skin measurement process 200 in which the skin parameters of the user may be measured in the physical location, such as in a store, in a lab or another other physical location. The user may participate in a user intake process 202, in which the user fills out an intake questionnaire that collects information, such as user preferences, life style and/or preferred form factors for the skincare products. The user intake process 202 may be done at the physical location or before the user enters the physical location. The user intake process 202 may be done online using a computing device like an iPad or orally by responding to questions. The questionnaire, for each user, may include, for example, questions assessing user skin concerns, user skincare product history, user self-reported skin needs, user sensorial preference, and user segmentation information. The questionnaire may produce a multivariate user profile consisting of preferences, perceived needs, and behavioral attributes. In addition, the questionnaire may, individually, or in combination with the set of skin parameters measured during the in person experience (the full biological skin measures) be used to produce a skin needs score for the six underlying skin need parameters as described above that represents the user's relative skin assessment against a normalized population of similar age, gender, and ethnicity. The skin assessment score creates a skin needs map that creates the relative skin need on the six skin health parameters, these scores and preference inputs may be used in the machine learning model to predict the optimal product and product regimen. The user may then do a skin physical process 204 in which the set of skin parameters (each of the skin parameters generated by the in person measurement process as described below) are captured. In one embodiment, the user may move between multiple measurement ‘stations’ individually, or be guided for measuring, informing, and consulting on skin analysis.

The user experience 200 may further comprise a sensorial or preference station process 206 and the sensorial station at the physical location may provide an interactive experience where users can explore formulation textures and fragrances to customize their formulations that can be analogized to wine tasting with a sommelier but for fragrance or textures. As described above, using the set of skin need parameters, the precision skincare process may determine the optimal skincare product formulation for the user.

The user in person experience 200 may generate optimal skincare product formulations during a custom formulations process 208 as discussed above in detail. Each optimal skincare product using the selected optimal skincare product formulation is produced. Each optimal skincare product is then delivered to the user on site, at home, or to another non-production site location.

The monitoring and optimization process 210 of the process 200 may generate a skin health dashboard for each user that is delivered to each user such as by interacting with the computing device as shown in FIG. 1A. An example of the skin health dashboard is shown in FIG. 6. The monitoring and optimization process 210 may also include remote monitoring of the skin of the user as part of the re-measurement and outcome data process described above.

FIGS. 2B1-2B3 illustrate further details of the in person measurement process that provides the user experience 200 shown in FIG. 2A. The skin physical 204 for the in person measurement may generate a dense skin parameter matrix 214 via in person measurements and a sparse skin parameter matrix 216 based on remote measurements. The in person measurement process may use one or more pieces of known measurement hardware (that may include just a physical device or elements (processor, etc.) or physical device or elements and software in the measurement hardware) to measure a set of skin parameters during the in person measurement. For example, the dense skin parameter matrix 214, as shown in FIG. 2B1, may include known spatial frequency domain imaging (SFDI) measures, confocal Raman spectroscopy measures, Courage & Khazaka measures, cross polarized and UV imaging, hyperspectral, and microbiome measures. The SFDI measures may include, for example, HbT1, superficial hemoglobin (papillary dermis), HbT2, subsurface hemoglobin (reticular dermis), a scattering amplitude, A, of the skin, StO2, (tissue oxygen saturation), melanin, diffuse reflectance and/or cross-polarized color photo. The following measures would be measured, deduced, and/or inferred from those measures: skin smoothness, blood capillaries/microvasculature of the skin, perfusion, pigmentation, collagen and/or color distribution and evenness of the skin. The Courage & Khazaka measures may include, for example, skin hydration, epidermal thickness, transepidermal water loss, skin pH and/or skin temperature. The confocal Raman spectroscopy measures may include, for example, pharmacokinetic (PK) profile across formulation vehicles via confocal Raman spectroscopy and/or a lipid profile of the skin. Pharmacokinetics describes what the body of a user does to an active ingredient and refers to the movement of the active ingredient into, in and out of the body including the time course of its absorption, bioavailability, distribution, metabolism, and excretion. The sparse skin parameter matrix 216 may include remote device measurements as described above.

Returning to FIG. 2B1, the set of skin parameters from the in person measurement (described above) may form a dense skin parameter profile matrix (the dense matrix) for users 1, . . . N (218) (the above discussed dense matrix) and the remote measurement may form a sparse skin parameter profile matrix for users 1, . . . N (222). As discussed above the full biological skin measures may be used to determine a skin need of the user. A dense skin profile inferred for each user from the sparse parameter profile matrix (224) as discussed above may be used as an input to a product matrix 226 shown in FIG. 2B2. The product matrix 226 may be generated by a machine learning predictive engine that characterizes active ingredients and delivery systems for skincare products for each user 1, . . . N. The machine learning process may also receive the dense skin parameter profile matrix as an input as shown in FIG. 2B2 and input from a skin care product ingredient database 228.

The product ingredients database 228 contains a plurality of ingredients for skin care products. Each ingredient may include active ingredients and inactive ingredients. Active ingredients are ingredients or compounds that are biologically active. Active ingredients, through one or more mechanism of action, interact with the biology to produce a desired change. Active ingredient examples include plant or algae extracts, pure compounds from vitamin esters, or synthetic peptides. Inactive ingredients are ingredients or compounds that are not biologically active and may include, for example, water, humectants, or emulsifying agents. These inactive ingredients may be included for sensorial or aesthetic properties. For the various active ingredients for skincare products, the product ingredient database 228 may correlate a machine learning prediction of efficacy for a particular skin need for each active ingredient. The product ingredient database 228 may be updated via the outcome data based learning inputs as described below. The product ingredient database 228 links a specific skin parameter (and/or the underlying measurement for the skin parameter), with one or more active ingredients and their delivery mechanism. In one embodiment, the system also has a learning loop where the above links and correlations in the product ingredients database 228 are tested to determine if the recommendation of active ingredient A for parameter measurement X in a skin profile of a user, provides a better outcome than active ingredient B for parameter measurement X. The product ingredient database 228 may be an expert system of curated relationships based on literature, clinical data, chemical structures, supplier data and/or previous experience with that particular active ingredient. Furthermore, outcome data of a user (after using the optimal skincare product with the selected one or more active ingredients and the selected delivery mechanism) may be generated by remeasuring the set of skin parameters of the user as described above and used to evaluate whether the predicted combination of active ingredient A for parameter measurement X is efficacious or inefficacious. Then, the system can update prior expectation to the posterior probability of seeing an efficacious outcome for that particular combination, leading to a new prior expectation for the next set of predictions thereby forming the learning loop.

The product ingredient database 228 avoids the cold start problem where there is no outcome data on the efficacy of a specific skin parameter/active ingredient combination from which to select the active ingredients for a user with a skin need. Specifically, since the product ingredient database 228 has specific skin parameter/active ingredient combinations regardless of the outcome data, it is still able to select specific skin parameter/active ingredient combinations for the user with the skin need. As the product ingredient database 228 is optimized with the outcome data so that there is a sufficient number of users and observed combinations, the framework can be extended to a multivariate case where more elements of the skin parameters and active ingredients are considered together. Furthermore, with a large number of users and outcomes, the system may use more black box solutions, like neural networks or mapping to most similar skin profile and skin need and selecting the active ingredient combination observed to deliver the best outcome for the skin needs of the user. In order to avoid getting the above selection process stuck in local minima or restrict particular combinations from being investigated some randomness is added to the selection of active ingredients similar to AB testing and/or multi-arm bandit testing. Thus, in essence a probability, P. is being calculating that is the P(outcome|profile, actives), which in the beginning (the cold start) can model as P(outcome|parameter, active) (as a singular parameter rather than the entire profile) and/or further P(OR>1|parameter, active)−the probability that we gain any benefit from treating a parameter at a certain value X with active A, where the comparator is the user's baseline measurement for said parameter.

Returning to FIG. 2B2, the observed outcomes measured via the full biological (dense) or remote (sparse) measures may be provided to a dense and sparse outcomes matrix (232) for each user 1, . . . , N as shown in FIG. 2B3. This outcomes matrix creates an outcomes based learning loop (230) that may be provided back to update the deep skin parameter profile (218), which, in turn, may be provided back to the correlation of dense to sparse (220) to update and inform the dense to expected outcomes and dense to sparse to expected outcomes correlation. Further, updates observed about ingredient efficiency based on the observed outcomes matrix (232) may also be input into the product ingredient database 228 as shown in FIG. 2B3. The product predictive engine 226 as described above with reference to FIGS. 4-1 and 4-2, generates custom skin care formulations 234 for each customer based on the skin parameter profile of each customer. Repeat measures, dense or sparse, create outcomes data for each specific pairing of formulation and skin profile 236. This outcomes measure of specific formulation to skin profile may then also feed into the outcomes matrix 232 as shown in FIG. 2B3. The output from the Dense & Sparse Outcomes Matrix 232 may be fed into the outcomes based learning loop (238) that in turn updates the inferred deep profile (224), the sparse skin parameter (222) the correlation of dense to sparse (220), which would, in turn update the custom formulations created 234, building a true learning model.

FIG. 3A illustrates the remote measurement user experience process 300. A user intake process 302 is the same as the user intake process described above and shown in FIG. 2A. During this user experience process 300, the user may then conduct a remote measurement and sensorial process 304. In one embodiment, a diagnostic and sensorial kit may be mailed/sent to the address provided by the user while in another embodiment, the diagnostic process may be delivered to the user to the computing device of the user. The diagnostic process may request biological/skin health diagnostics using a remote measurement device (that may be part of the computing device of the user or a separate device) that, as described above captures a RGB image of the skin of the user. The data generated by the diagnostic process may be sent back to the backend 104 via the computing device of the user. As a result, the user does not need to come to the physical location for the in person measurement since the full biological measurements can be inferred as described above.

Like the in person measurement user experience process 200 above, the remote measurement user experience process 300 gathers the different skin parameters. However, since those skin parameters are sparse (due to the remote measurements), their dense skin parameters are inferred using, previously measured paired measurements of dense to sparse skin parameters as described above. The remote measurement user experience process 300 may perform the same selection of active ingredients and delivery mechanisms as described above to generate the optimal skincare formulation for the user except that the inferred dense skin parameters are input to the product ingredients matrix (and the machine learning processes) to select the one or more active ingredients and the delivery mechanism.

The remote measurement user experience process 300 in FIG. 3A may then generate optimal skincare formulations customized to the user 306, produce the optimal skincare products for the user and deliver the optimal skincare products to the user that is the same as described above for the in person measurement user experience. The remote measurement user experience process 300 also includes a monitoring and optimizing process 308 that is the same as that process described above except that this process 308 also uses the outcome data to build empirical sparse measures to replace and/or augment inferred deep biological profile as an input to ML model, and to update profile/parameter to active ingredient mechanism of action. This process also updates the ongoing formulations described above. Thus, whether the process is in person or remote, the precision skincare process gathers the relevant skin parameters for the user, selects optimal skincare product formulations using machine learning/AI and performs the monitoring and optimization so that the optimal skincare product formulation selections are being continuously updated and optimized for each user to handle changes to the user's skin parameters and/or user preferences changes and/or changes due to the efficacy/lack of efficacy of a particular active ingredient for a particular skin need.

FIGS. 3B1-3B4 illustrate more details of the remote measurement process 300 that also incorporates some elements of the in person measurement process. Elements and processes with similar reference numbers (212, 214, 216, 218, 220, 222 and 224 in FIG. 3B1) are the same and operate in the same manner as described above and thus will not be described further here. In addition to those processes, empirical sparse measures (238) from the remote testing/measurements may be used to update the sparse to dense correlation model 222 used to infer the set of skin parameters in the dense matrix (220). As shown in FIG. 3B3, the remote measurement process may include a process 312 in which the empirical sparse profile 310 has been constructed from empirical sparse to outcomes measures from 232 and updated via the learning loop that replaces or augments inferred dense skin parameter matrix as an input to the ML model updates. When the user 212 performs remote measurement of the skin parameters and without requiring inference of the dense profile (220, 218), the empirical sparse profile 310 is used to generate the optimal skincare product customized formulation for the user based on sparse measures alone.

The correlation process 220 in FIG. 3B1 may correlate the sparse skin parameters captured remotely to the dense skin parameters. In one embodiment, the correlation may be performed using a machine learning process. In more detail, the correlation process 220 enables the optimal skincare product customized formulations via sparse (remote) data measurement by 1) initially inferring the dense biological profile and 2) at scale using outcomes based learning models to build an empirical outcomes model correlated to the sparse measurements as described above.

FIGS. 3B1 and 3B2 also show that the sparse measures 302 is generated by a sparse skin parameter profile matrix (222) for each user 1, . . . , N. The sparse skin parameter profile matrix (222) is trained and updated using a learning process 350 with inputs from processes 310 (the empirical sparse skin profile matrix) and 312 (shown in FIG. 3B3) based on the outcome data.

As shown in FIGS. 3B2 and 3B3, one of the inputs to the sparse skin profile in FIG. 3B2 as an empirical spare outcomes measures learning loop 312 that builds empirical sparse measures to replace and/or augment inferred deep biological profile as an input to the machine learning process model and the sparse outcomes matrix for each user 316 as shown in FIG. 3B4. The empirical sparse skin profile matrix 310 may be fed into a correlation process 314 in which the sparse measures are correlated directly to the sparse/empirical outcomes measures and used for input to replace/augment the deep profile inferences 224 into the product matrix 226 as shown in FIG. 3B1-3B3. As before, the custom formulas 234 are generated using the matrix 226 in which repeat measurement process 318 updates the sparse outcomes matrix 316 for each user. In one embodiment, the sparse outcomes matrix may be a reconstructed hyperspectral data cube, but the sparse measures may also be obtained from a color metric assay sent to the user and the like. The output from the sparse outcomes matrix 316 may be fed into an outcomes based learning loop 320 based on the outcomes, the sparse skin measures, skin profile, ingredients and formulations to inform and update the machine learning methods and fed back to the product matrix 226. Further, the output of the sparse outcomes matrix 316 may be fed into an outcome based learning loop 322 that update the dense to sparse model expected outcomes with empirical outcomes that are fed back to the conversion process 220 in FIG. 3B1.

As shown in FIG. 3B4, the output from product matrix 226 may be a custom formula 340 that is fed into the re-measure process (either in person or remote re-measure) 318. The output of that remeasuring process 318 may be fed into a dense and sparse outcome matrix 342 that contains outcome data for each user. The outcome data from the dense and sparse outcome matrix 342 may be fed back, via an outcome learning loop to the dense skin parameter profile matrix 218 as shown in FIG. 3B1. The outcome data updates the joint distribution between skin profiles and active ingredients in the dense skin parameter model 218. In addition, the outcome data from the dense and sparse outcome matrix 342 may be fed back, via an outcomes learning loop to the product matrix 226 as shown in FIG. 3B3. In this outcomes learning loop, the skin measures, skin profile, active ingredients and formulations inform and update the machine learning ingredient/skin need/skin profile matrix. In addition, the outcome data from the dense and sparse outcome matrix 342 may be fed back, via an outcomes learning loop to the ingredient database 228 as shown in FIG. 3B3 that updates observed (from the outcome data) mechanism of activation (MOA) and ingredient efficacy.

FIGS. 4-1 and 4-2 show a particular implementation of a predictive process 400 that may be used to generate the optimal skincare product formulation customized for a user. However, it is understood that the precision skincare process may use other specific predictive processes that are all within the scope of the disclosure. The process 400 shown in FIGS. 4-1 and 4-2 is performed for the in person measurement process and the remote measurement process described above. As shown in FIGS. 4-1 and 4-2, a database of ingredients (including active ingredients) for skin care products 402, skin care supplier data 404 (that may include primarily clinical efficacy studies for skin care products, benefit and a description of mechanism of action and/or functional skincare benefit 406 (discussed in more detail in the example) are input into a predictive model 408. The predictive model 408 may be a natural language processing (NLP) model that maps the data of ingredients to the skin parameters of the particular user. The predictive model 408 may comprise of multiple forms, as an expert system, a Bayesian optimization model, a deep neural network, support vector machine and/or a hybrid model encompassing multiple learning models. In one example, the skin parameters of the user (generated from the dense matrix and the sparse matrix) may include hydration/skin barrier, skin smoothness, skin tone, skin milieu, oxygen saturation, dermal fibers (all skin biological parameters) and texture, feel, scent, allergen, production route, trade name and/or mixture component (particular user preferences and lifestyle). The user skin parameters may then be mapped for each ingredient of the skin care products. In some embodiments, the system may generate the user interface for each parameter that is shown in FIGS. 4-1 and 4-2.

As further shown in FIGS. 4-1 and 4-2, the prediction method 408 may first select the one or more active ingredients and delivery mechanism to address a primary skin need of the particular user. The primary skin need of the particular user is based on the set of skin parameters and may be the skin need that is farthest away from a normal and/or healthy skin profile. The prediction method 408 may then also select the one or more active ingredients and delivery mechanism to address the secondary skin needs of the particular user.

The prediction engine may include a learning model 408A of skin parameter measurements to outcomes based on evidence of ingredient function that can later also include user based outcomes linked to skin biology of actual users of the system so that the model learns through feedback and will become more accurate in its recommendations. The user based outcomes used to improve this model may be fed back (408B) based on each user outcome linked to one or more skin parameters based on the optimal skin care product formulations customized and provided to each user.

The precision skincare process may include at least three learning loops used with outcomes data and each learning loop enables three distinct key elements of our precision system to be updated, and deliver more accurate results with scale. First, based on the re-measurements after usage of a given combination of actives, the system gains more information of whether the predicted combination is efficacious for a given skin parameter/profile. The method is therefore able to form a joint probability distribution over skin parameters and active ingredients relative to outcomes that enables future predictions of potential outcomes. During this learning loop, the method is learning for both individual active ingredients, and from the final full product (a combination of active ingredients and a specific vehicle). The singular learnings on active ingredients will confirm mode of action and effect on skin phenotype, while the combinatorial formulation will enable insights into potential synergistic/antagonistic effects between specific active ingredients, and/or skin profiles. For instance, a good effect of hyaluronic acid for skin hydration in a given profile may be seen, but a lesser effect in another profile if that profile has a skin barrier issue and are therefore not able to hold water in the skin due to evaporation. Then fixing this issue by building a stronger barrier will lead to a better skin hydration—there is therefore a combinatorial interplay between skin profiles and actives.

The second learning loop, as the method measures both hyperspectral and RGB images in the in person measurement process, better enable further learning on the reconstruction of hyperspectral images from RGB. In addition, since the method is doing matched samples of hyperspectral and full skin profiles, it also enables inference of the dense biological matrix from the hyperspectral data, and via a two-step inference model it enables the RGB to hyperspectral to dense biological matrix inference thereby enabling the remote measurement process.

The third learning loop handles noisy inferences. In particular, in the remote measurement process, the method is doing re-measurements with the RGB camera and inferring the hyperspectral data which means repeat measurements on a likely noisy inference. However, the noise will be deterministic and thereby creating a systematic pattern that enables a learning loop directly based on inferred hyperspectral data, product recommendation, and re-measurements. More specifically, the learning loop based on outcomes reinforces the selection of the active ingredients for a given skin need. The learning loop also reinforces a reconstruction of the full biological skin profile from a hyperspectral image, and the reconstruction of the hyperspectral image from an RGB image that provides the ability to use the remote measurement process. In the remote measurement process, there is also a learning loop that learns based on the outcomes measured via this reconstruction which may or may not be perfect, and therefore could deviate from the learning loop in the in person measurement process.

Referring back to product matrix 226 in FIG. 3B3, in one implementation of the system and method, the machine learning processes may be based on OLS, deep learning and the Bayesian framework. Overall, the learning model has a digital and analog phase. The digital being the prediction phase from measurement to ingredient combination, and the analog being the time it takes the user to use up the recommended product and get back for a re-measurement to update the system. In the example, assume that we have user1. At time t0 the system only contains initialization parameters given by the curated expert rule based system. User1 is then given a product based on the rules linked to each particular measurement. Between user1's first visit and revisit at t1 the system sees a series of other profiles from user2 . . . userN, some of which will have revisits and new measurements which are then outcome parameters for a specific measurement/ingredient combination. Upon revisit of user1 the new set of measurements are used in two ways. One to form user1 specific outcomes, and two, to update the overall measurement/ingredient/outcome linkages. The new measurements are then our new prior for which we need to find an ingredient combination.

In the OLS/PLS framework, the new measurement from user1 is regressed on the profiles of user2, . . . , userN to find the coefficients of the linear combination. These coefficients are then used against the ingredient/outcome profiles of user2, . . . , userN to find the new recommendation for user1. In the deep learning framework, baseline and outcome measurements are provided as inputs and the linked ingredient combination is the output. The model will then start to learn associations between deltas in measurements and specific ingredient combinations. In a Bayesian framework, the method can treat each ingredient as being equally likely to confer a beneficial or a detrimental outcome, upon observing the link between a given skin parameter and a given outcome for an ingredient we update our belief of the outcome. This new ‘belief’ then becomes the prior for new recommendations. Using such a sequential trial design allows for continuous learning where the method, at any given time, can calculate all the posterior probabilities for probability of any beneficial effect, probability of a clinically relevant effect, probability of harm and/or probability of similarity of outcomes between different treatments.

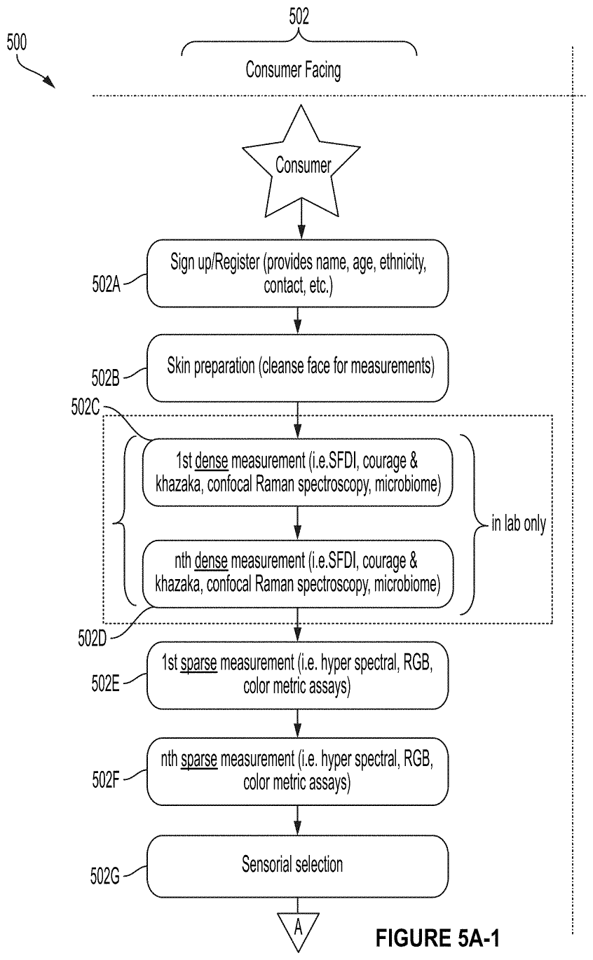

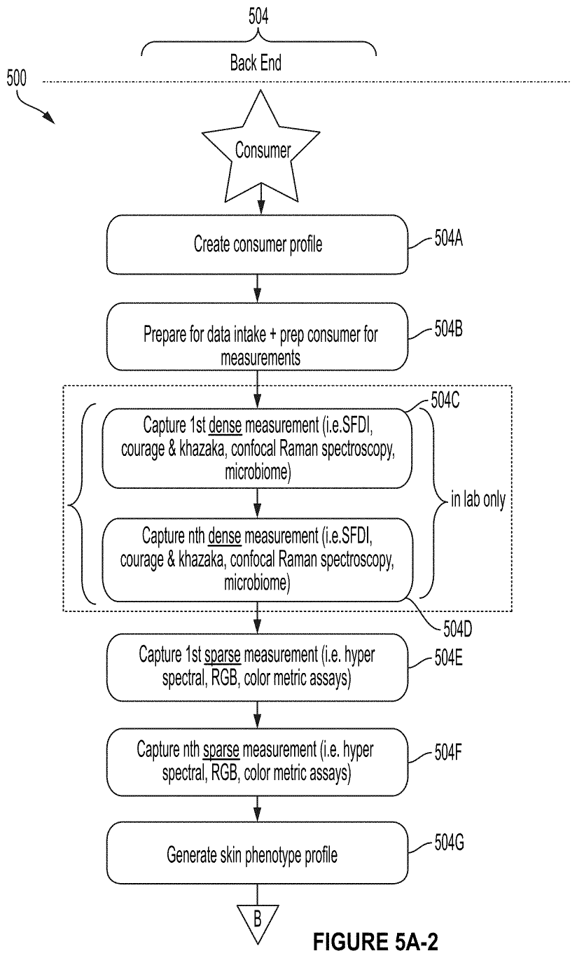

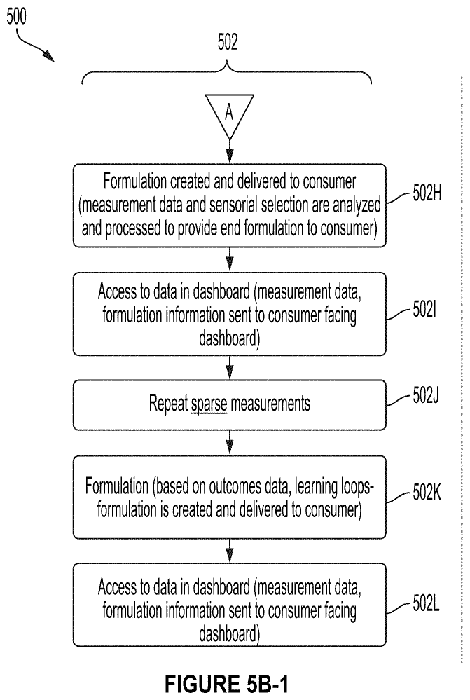

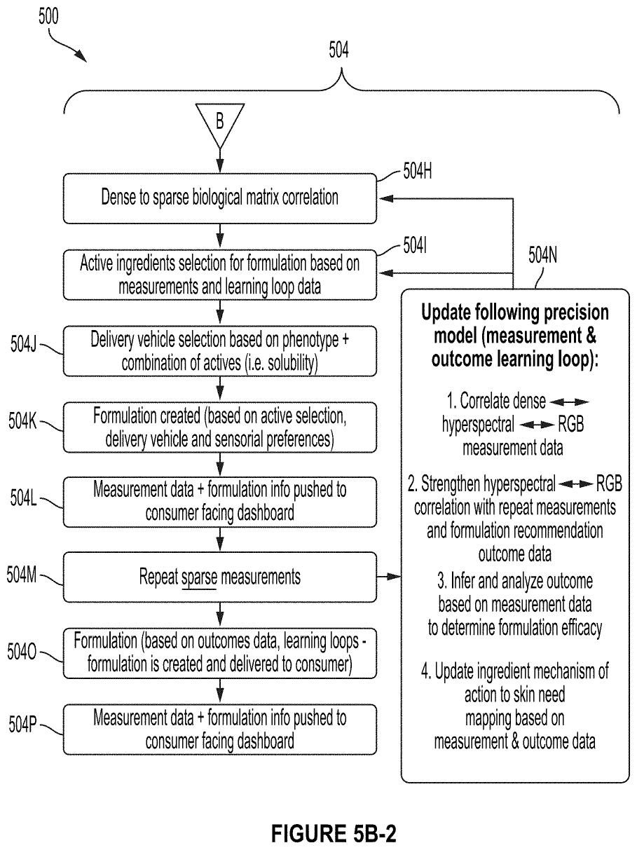

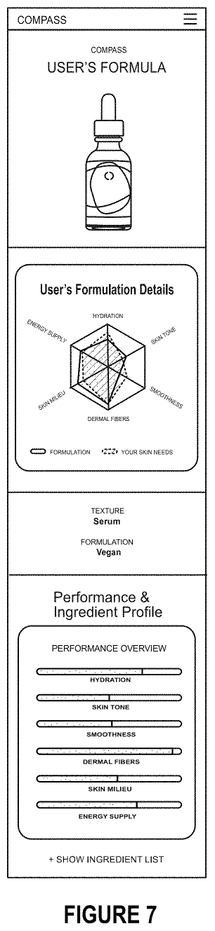

The above descriptions have focused on the processes for each of the in person and remote measurement processes regardless of how those processes were performed. FIGS. 5A1-5A2 and 5B1-5B2 illustrate the overall precision skincare process 500 divided into user facing processes 502 and backend processes 504 performed by the method. For the user facing processes for each user, each user may sign up and register for the precision skincare (502A) and may then perform skin preparation for the measurements (502B) such as cleaning his/her face. The user may then take the measurements and then, certain measurement analysis processes 502C, 502D may be performed in a physical location including the first dense measurement (502C) and any subsequent dense measurements (502D) using the measurement hardware described above. Then, a first sparse measurement (502E) and any subsequent sparse measurements (502F) may be performed using the measurement hardware described above. A sensorial selection (502G) may then be performed. Based on the dense and sparse measurement and the machine learning processes discussed above, a skin care formulation (502H) for each user is created and delivered to the user in which measurement data and sensorial data are analyzed and processed to provide the formulation with examples of that user interface shown in FIGS. 6 and 7 which are described below in more detail. The user also now has access to the data in a dashboard (5021) in which the data includes the measurement data, the formulation information, examples of which are shown in FIGS. 6-7. The method may now perform optimization and feedback processes and, for example, repeat the sparse measurement (502J) using the remote monitoring as described above so that the formulations for the user may be adjusted if/when the skin profile of the user changes or to improve the formulation based on later discovered information or later outcomes from other users of the system. Then, formulations (based on outcome data, learning loops) are generated and delivered/displayed to the user (502K) along with access to the dashboard with the updated information (502L).

The backend processes 504 may include creating a profile for each user (504A) and then prepare for data intake and user measurements (504B). The backend may then capture the first dense measurements (504C) and any subsequent dense measurements (504D) using the measurement hardware described above. Then, a first sparse measurement (504E) and any subsequent sparse measurements (504F) may be captured using the measurement hardware described above. The backend (including the machine learning processes described above) may generate a skin profile for the user (504G) and generate the dense to sparse biological matrix correlation (504H). The backend then selects the active ingredients for each formulation based on the skin profile and measurements and learning loop data (5041) and selects the delivery vehicle (504J) and sensorial preferences. The backend then generates custom formulations for the user (504K). The backend then pushes (504L) the skin measurement data and formulation(s) to the dashboard of the particular user. The method may now perform optimization and feedback processes and, for example, repeat the sparse measurement (504M). The backend may then perform an update for the user using the precision skincare model using the updated skin measurement data and the outcome learning loops as described above. Then, formulations (based on outcome data, learning loops) are generated and delivered/displayed to the user (5020) along with access to the dashboard with the updated information (502P).

FIGS. 6 and 7 illustrates example of the user interface of the skincare system showing a recommended formulation and skin profile for each user. In particular, FIG. 6 shows the dashboard with the user unique and specific skin profile using the skin parameters as well as the skin health score relative to other users. The dashboard data is based on the measurements taken and the analysis by the system. FIG. 7 shows the recommended skincare product. The user interface in FIG. 7 may also show the formulation details based on the different skin parameters with the skin needs for the particular user and the formulation results. The user interface may also have a performance overview that shows how well the custom formulation addresses the consumer's skin needs as a progress bar, spider graph or other such visualization to show the ingredients of the custom formulation as a fit for the consumer's skincare needs.

In some embodiments, in addition to the above selection of active ingredients based on skin profiles and skin needs, the system may also customize based on pharmacokinetics as the absorption across skin has been shown to have a high inter-individual variance. In particular, once the system has predicted the optimal active ingredient, or active ingredient combinations, for a given user, the system may predict the optimal delivery system to enhance absorption of the active ingredients and/or transport the selected active ingredients across the skin barrier of the given user. To handle this absorption variability, the system may be used to formulate a number, such as 4-8, different delivery mechanism vehicles with different physicochemical properties and use confocal Raman spectroscopy to measure the absorption rates in an individual across the different formulations. This measurement will allow the system to assess skin profiles and skin needs correlated with specific absorption rates for each formulation and build a predictive model that can infer the absorption rates from the skin parameter measurements. The system can then provide the optimal skincare product formulation with the best absorption profile for any given skin profile or the optimal skincare product formulations for which the system has established a predictive model based on empirical data.

### User Feedback Measurement

The system may include a mobile based application integrating dashboard reporting, tracking, etc./RGB to hyperspectral analysis tool/capturing skin data using processes to infer from dense skin parameter data matrix. Thus, the individual user has a digital application (executed by a processor of the computing device of the user as a plurality of instructions that configure the processor) available via their computer, tablet, mobile phone (native or web-based), etc. which the user can use to do various actions. For example, the digital application allows the use to capture and submit skin measurements (to generate the sparse matrix), view their skin parameter measurements and analysis (see FIG. 6 for example) and/or track and monitor progress with respect to their skin parameter measurements and analysis (also shown in the user interface of FIG. 6). The mobile application may also provide the user with access to tools, tips and prompts for repeat measurements, access to formulation information and formulation history (ingredient list, ingredients info), access to feedback/reporting tools for skin progress, formulation, reactions, etc. and access to account info/onboarding to collect preferences, demographic details, billing. In one implementation, the mobile application may be based on Elixir/Erlang as a primary programming language for back-end, PostgreSQL as a persistence layer and RabbitMQ as a message broker, JavaScript as a primary programming language for front-end. Knowledge base is built with multi-stage concurrent data ingestion and processing pipelines with support of back-pressure, fault tolerance and graceful shutdowns.

### Skin Care Recommendation Use Case Customer Perspective Example

The measurements from a single user may generate a deep biological and a sparse biological skin profile as discussed above. Based on this profile a personalized combination of ingredients for the skincare are formulated. Through repeat measurements either in person or remotely or remeasures the efficacy of that particular combination of ingredients for that particular skin profile of the user will be assessed. Across multiple users, the system is able to map each individual skin profile to similar skin profiles and compare the outcomes from their personalized skincare products, and update initial recommendations to those that were most efficacious for others with a similar profile. With increased data volumes, the system is able to shift the recommendations based on similarity measures and/or simple ordinary least squares/partial least squares regression to more black box solutions based on deep neural networks. For the remote model, since the system establishes a link between the sparse skin profile and the deep skin profile in a user specific manner, the system is able to use similar mapping strategies to link remote sparse measurements into the deep skin profiles and again into ingredient and outcomes measures. Since the remote model is also based on re-measurement over time, the system establishes an empirical validation of the skincare predictions and can change recommendations based on the feedback from subsequent measurements.

### Example of Precision Skincare Method

Consumer A signs up for the precision skincare services provided by the system shown in the Figures. After completing her or his registration, she or he arrives at the Compass Beauty Inc skincare store for her or his skin bio assessment. At the store she or he is greeted by the bio associate who will take her or him through the measurement experience across the specific measurement stations that may generate her dense biological skin-profile (dense matrix).

Consumer A's measurement at the Courage and Khazaka station shows that she or he has a low hydration parameter. Specifically, they score 34 on the corneometer (low) which assesses the superficial hydration and 15 (high) on the high trans-epidermal water loss (TEWL). The low superficial hydration measure and high TEWL measure is indicative of a skin barrier defect as the root cause of her or his low hydration so the expert system (using the model, etc. as described above) recommends that something should be done to improve the skin barrier, and thereby water retention in the skin for Consumer A.

The ingredient database contains specific ingredients with known clinical benefits and mechanisms of actions and how these mechanisms are tied to individual skin parameters and the expert system evaluates active ingredients known to improve epidermal thickness and thereby benefit a disrupted skin barrier. Consumer A's skin profile is assessed to be similar to other skin profiles (stored in the system) that have been observed to respond most effectively to bakuchiol that is an active that improves epidermal thickness and skin barrier. Therefore, bakuchiol is selected by the expert system for the skincare product customized for Consumer A.

Consumer A's measurement via spatial frequency domain imaging (SFDI) indicates a scattering coefficient which is suboptimal and in the 30^{th} percentile of her or his peers. The scattering coefficient is linked to the elastin/collagen matrix. Thus, the expert system recommends an active that is known to improve the elastin/collagen matrix. Given Consumer A's skin profile, it is predicted she or he will see the best results with a mixture of Acetyl Tetrapeptide-2 and Caprylyl Glycol.

Consumer A is measured across all six parameters evaluating her individual skin needs and the expert system, like for the first two parameters, evaluates the optimal formulation for that parameter and in combination with the other parameters and needs.

Consumer A's precision skincare formulation is created based on the active combinations and levels selected by the expert system. She receives her precision skincare formulation with among other ingredients, a high concentration of bakuchiol for skin barrier and a mixture of Acetyl Tetrapeptide-2 and Caprylyl Glycol for elastin regeneration.

Consumer A proceeds to use her skincare formulation daily for a predetermined period of time, such as 4 weeks in this example. At the end of the predetermined period of time, Consumer A returns to the store and has her or his skin parameters measured. In this measurement, it is noted that her or his superficial hydration is now 40 and TEWL is 12. Both have improved but need further improvement for optimal skin health and appearance. The expert system again recommends her formulation contain bakuchiol to continue her skin barrier improvement. It is also assessed, using the SFDI, that her elastin collagen matrix scattering coefficient is still lower than that of her peers and not improved as expected during the first treatment period. The expert system updates the outcomes/prediction model for her skin profile to reflect that Acetyl Tetrapeptide-2 and Caprylyl Glycol was not as effective as predicted for elastin collagen rejuvenation in this skin profile. The system then selects a mixture of Butylene Glycol, Carbomer, Coco-Glucoside, Palmitoyl Tripeptide-1, and Palmitoyl Tetrapeptide-7 that also benefits the elastin collagen matrix but through a different mechanism of action. Consumer A's new precision skincare formulation is then created based on the new measurements, outcomes, and predictions. She or he receives her new formulation and uses the formulation again for a predetermined period of time, such as four-weeks.

After the predetermined period of time, Consumer A again is requested to remeasure her skin parameters to assess skin need progress for Consumer A and create her or his next treatment skincare formulation. This time, Consumer A does not go in store, but instead, using the camera in her or his smartphone, consumer A takes a remote measurement. Consumer A captures several pictures of her or his skin based on prompts from an application running on the smartphone. The RGB image captured on the smartphone is used to reconstruct a hyperspectral image which is used to infer her or his dense biological matrix. This is possible due to the linked dense biological measurements, hyperspectral images and RGB images taken in the physical experience, thereby creating the mapping needed to learn the inverse inference algorithm.

Based on the inferred dense skin profile, the expert system determines that consumer A has an improved TEWL measure of 9 (now inferred, and not directly measured). This observation (outcome data) leads the ingredient recommender system (the database and model are updated using a machine learning loop) to strengthen the association between TEWL and bakuchiol as a beneficial combination and a beneficial combination for Consumer A's skin profile and similar skin profiles. If the system had determined the opposite, a status quo or further increase in TEWL, the association between TEWL and bakuchiol as a beneficial combination would have been lowered. This dynamic changing of the association between measurements and ingredients for a beneficial, detrimental or no effect outcome is continuously updated across the consumer population leading to a learning model, which with increase scale, can be partitioned into finer segments based on demographic characteristics of the user and later into specific skin profiles. As a result of these updates, the system then already contains outcome data for a closely matched profile and a specific ingredient combination.

Based on her remeasures, consumer A now has a normalized skin barrier as inferred from the TEWL, which again was inferred via an inferred hyperspectral image from a RGB image. Similarly based on the remeasures, it is clear that the next priority skin issue to optimize for Consumer A is evenness of skin tone as that parameter is most reducing (negatively affecting) her or his overall radiance. The new skincare formulation consumer A receives is now based on these new remeasures with ingredients selected and optimized as described above to maximize her overall skin radiance and the individual skin parameters. Remeasures continue over time in physical or remote settings and the above learning cycle is repeated.

Consumer A recommends the precision skincare solution to her or his sister, Consumer B, who then signs up. Consumer B completes her registration on her mobile phone, inputting information about her preferences, skin history, and needs. After her registration is complete, she elects to take her skin measurements via the camera on her mobile device instead of taking them in store. She opens the application, captures the images of her skin as instructed in the application and sends her skin assessment and data. The RGB image captured on the mobile phone is used to reconstruct a hyperspectral image. The reconstructed hyperspectral image and data cube is then used to infer Consumer B's dense biological measurements from the previously linked dense to sparse biological measurements. As in the above example, this is enabled by an inverse inference learning algorithm.

Based on the inferred dense skin profile, we see that consumer B, has a (high/negative) TEWL measure of 12 (now inferred, and not directly measured). Further it is observed that Consumer B has a similar consumer profile as Consumer A. Given this similar profile, the ingredient recommender system recommends bakuchiol for Consumer B given the observed prediction and outcome of bakuchiol in improving Consumer A's high TEWL score. Consumer B's skin is assessed on the other parameters and a skincare formulation recommendation is generated. She receives an individually personalized formula containing bakuchiol among other ingredients recommended for her specific skin needs and profile. Consumer B then uses the product and after one month then remeasures her skin using the camera on her mobile phone to generate her new formulation. Based on Consumer B's remeasured TEWL state the relationship between TEWL, bakuchiol and this skin profile will be further updated, continuing the learning process.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

1. A precision skincare method, comprising:
capturing a first set of skin parameters of a user, wherein the first set of skin parameters includes surface and sub-dermal parameters and wherein capturing the set of skin parameters comprises hyperspectral imaging and/or a hyperspectral data cube;
determining a first dense skin parameter matrix based on the hyperspectral imaging and/or hyperspectral data cube;
determining a skin need of the user based on the first dense skin parameter matrix;
providing a database that has a plurality of entries wherein each entry has an ingredient of a skincare product or a skin need associated with what is known to be improved by the ingredient and a model that accesses the entries in the database;
formulating a skincare product customized for the user based on one or more of a selected active ingredient and a selected delivery mechanism for the determined skin need of the user, wherein the one or more active ingredient ingredients and delivery mechanism are selected using a machine learning process and the model;
capturing a hyperspectral image or a red-blue-green (RBG) image of the user after use of the skincare product customized for the user;
inferring a second set of skin parameters from the hyperspectral image or RBG image;
determining a sparse skin parameter matrix and/or a second dense skin parameter matrix from the second set of skin parameters;
generating outcome data after use of the skincare product customized for the user based on the sparse skin parameter matrix and/or the second dense skin parameter matrix;
updating the model for each piece of user outcome data including the generated outcome data for the user;
optimizing the selection of one or more of the active ingredient and the delivery mechanism for the user using the machine learning process and the updated model; and
generating an updated skincare product for the user using the optimized selection of the one or more of the selected active ingredient and the delivery mechanism.

2. The method of claim 1, wherein capturing the first set of skin parameters further comprises capturing the first set of skin parameters during an in person measurement process.

3. The method of claim 2, wherein capturing the first set of skin parameters during the in person measurement process further comprises using spatial frequency domain imaging hardware, hyperspectral imaging hardware, red-green-blue (RGB) imaging hardware, confocal raman spectrometry hardware, imaging using UV, cross polarized, or parallel polarized light, and probes.

4. The method of claim 3, wherein the first set of skin parameters comprises a hydration parameter, a skin tone parameter, a smoothness parameter, a dermal fiber parameter, a skin milieu parameter and an energy supply parameter and wherein generating the dense skin parameter matrix further comprises populating the dense skin parameter matrix with the hydration parameter, the skin tone parameter, the smoothness parameter, the dermal fiber parameter, the skin milieu parameter and the energy supply parameter.

5. The method of claim 4, wherein capturing the hyperspectral image or the RBG image further comprises capturing the hyperspectral image or the RBG image during a remote measurement process and generating a sparse skin parameter matrix for the user.

6. The method of claim 5, wherein capturing the RBG image during the remote measurement process further comprises capturing a red-green-blue (RGB) image of the skin of the user using a camera of a computing device of the user.

7. The method of claim 1, wherein selecting the one or more active ingredients further comprises determining a probability of a skincare outcome based on the skin need of the user, efficacy of one or more selected active ingredients and outcome data already stored in a product ingredient matrix.

8. The method of claim 1 further comprising producing the optimized skincare product having an optimal formulation based on the selected one or more active ingredients and the selected delivery mechanism and delivering the produced optimal skincare product with the optimal formulation to the user.

9. The method of claim 1, wherein updating the model with the outcome data further comprises updating a product ingredient matrix with the outcome data.

10. The method of claim 1 further comprising determining an overall skin health of the user based on the first dense skin parameter matrix, second dense skin parameter matrix or the sparse skin parameter matrix of the user.

11. The method of claim 1, wherein determining the skin need further comprises comparing the first dense skin parameter matrix, second dense skin parameter matrix or the sparse skin parameter matrix of the user to an ideal dense skin parameter matrix or sparse skin parameter matrix to determine the skin need of the user.

12. The method of claim 1, wherein updating the model for each piece of user outcome data further comprises updating the model for all users.

13. The method of claim 1, wherein providing the database further comprising providing an entry for an active ingredient and an entry for an inactive ingredient.

14. The method of claim 1, wherein the second set of skin parameters further comprises a hyperspectral cube reconstructed from the skin of the user.

15. A precision skincare system, comprising:
one or more pieces of measurement hardware;
a computer system connected to the one or more pieces of measurement hardware, the computer system having a processor and memory and a plurality of lines of instructions wherein the processor of the computer system is configured to:
receive a first set of skin parameters of a user having a predetermined number of skin parameters of the user, wherein the first set of skin parameters includes surface and sub-dermal parameters and wherein capturing the set of skin parameters comprises hyperspectral imaging;
determining a first dense skin parameter matrix based on the first set of skin parameters;
determining a skin need of the user based on the first dense skin parameter matrix;
provide a database that has a plurality of entries wherein each entry has an ingredient of a skincare product or a skin need associated with what is known to be improved by the ingredient and a model that accesses the entries in the database;
formulating a skincare product customized for the user based on one or more of the selected active ingredient and selected delivery mechanism for the determined skin need of the user, wherein the one or more active ingredient and delivery mechanism are selected using a machine learning process and the model;
receive a a hyperspectral image or red-blue-green (RBG) image of the user after use of the skincare product customized for the user;
using the hyperspectral image or RBG image to reconstruct a hyperspectral cube; inferring a second set of skin parameters from the hyperspectral cube;
determining a sparse skin parameter matrix or a second dense skin parameter matrix from the second set of skin parameters;
generate outcome data after use of the skincare product customized for the user using the sparse skin parameter matrix or second dense skin parameter matrix;
update the model for each piece of user outcome data including the generated outcome data for the user;
optimize the selection of one or more of the active ingredient and the delivery mechanism for the user using the machine learning process, the updated model, the sparse skin parameter matrix and/or the second dense skin parameters matrix; and
generate an updated skincare product for the user using the optimized selection of one or more of the selected active ingredient and the delivery mechanism.

16. The system of claim 15, further comprising one or more additional pieces of measurement hardware connected to the computer system that capture the first set of skin parameters of a user.

17. The system of claim 15, wherein the processor is further configured to receive the first set of skin parameters during an in person measurement.

18. The system of claim 16, wherein the one or more pieces of measurement hardware are spatial frequency domain imaging hardware, hyperspectral imaging hardware, red-green-blue (RGB) imaging hardware, confocal raman spectrometry hardware, imaging using UV, cross polarized, or parallel polarized light, and probes.

19. The system of claim 18, wherein the first set of skin parameters comprises a hydration parameter, a skin tone parameter, a smoothness parameter, a dermal fiber parameter, a skin milieu parameter and an energy supply parameter and wherein the processor is further configured to populate the dense skin parameter matrix with the hydration parameter, the skin tone parameter, the smoothness parameter, the dermal fiber parameter, the skin milieu parameter and the energy supply parameter.

20. The system of claim 19, wherein the processor is further configured to receive the hyperspectral image or RBG during a remote measurement and infer a second set of skin parameters for the user.

21. The system of claim 19, wherein the (RGB) image of the user is captured using a camera of a computing device of the user.

22. The system of claim 15, wherein the processor is further configured to determine a probability of a skincare outcome based on the skin need of the user, efficacy of one or more selected active ingredients and outcome data already stored in a product ingredient matrix.

23. The method of claim 15, wherein the processor is further configured to update a product ingredient matrix with the outcome data.

24. The system of claim 15, wherein the processor is further configured to select, using the machine learning process and the provided model, an optimal one or more active ingredients and the amount of each active ingredient for the skin need based on the skin need.

25. The system of claim 15, wherein the processor is further configured to determine an overall skin health of the user based on the measured first dense skin parameter matrix, second skin parameter matrix or sparse skin parameter matrix of the user.

26. The system of claim 15, wherein the processor is further configured to compare the measured first dense skin parameter matrix, second skin parameter matrix or sparse skin parameter matrix of the user to an ideal first dense skin parameter matrix, second skin parameter matrix or sparse skin parameter matrix to determine the skin need of the user.

27. The system of claim 15, wherein the processor is further configured to update the model for all users.

28. The system of claim 15, wherein the processor is further configured to provide an entry in the database for an active ingredient and an entry in the database for an inactive ingredient.

29. The method of claim 1, wherein the hyperspectral image or RBG image is used to reconstruct a hyperspectral cube.
